(19) United States
(12) Patent Application Publication
(10) Pub. No.: US 2023/0086571 A1
(43) Pub. Date: Mar. 23, 2023

(54) METHODS AND DEVICES FOR LEAFLET FOLDING OR CAPTURE

(71) Applicant: Edwards Lifesciences Corporation, Irvine, CA (US)

(72) Inventors: Ofir Witzman, Harish (IL); Eitan Atias, Netanya (IL); Hanoch Cohen-Tzemach, Holon (IL); David Maimon, Atlit (IL)

(21) Appl. No.: 18/058,708

(22) Filed: Nov. 23, 2022

Related U.S. Application Data
(63) Continuation of application No. PCT/US2021/034409, filed on May 27, 2021.
(60) Provisional application No. 63/031,056, filed on May 28, 2020.

(51) Int. Cl. A61B 17/122

(52) U.S. Cl. CPC A61B 17/122; A61B 2017/00349

(57) ABSTRACT

Methods and tools for folding and/or capturing leaflets of a heart valve are disclosed herein. Prior to or during installing of a prosthetic heart valve, part of a leaflet of an existing valvular structure can be positioned distally and/or folded upon itself. The existing valvular structure may be a native heart valve or a previously-implanted prosthetic heart valve. When the existing valvular structure is at the aortic position, the prosthetic heart valve can be subsequently installed within the existing valvular structure such that the leaflet is maintained in a location that avoids obstructing blood flow to one or more of the coronary arteries.

### CROSS-REFERENCE TO RELATED APPLICATION

This application is a continuation of PCT patent application no. PCT/US2021/034409, filed May 27, 2021, which application claims the benefit of U.S. Provisional Application No. 63/031,056, entitled “Methods and Devices for Leaflet Folding or Capture,” filed May 28, 2020, each of these patent application being incorporated by reference herein in its entirety.

### FIELD

The present disclosure relates to prosthetic heart valves, and to methods and devices for folding or capturing leaflets of existing valvular structures prior to or during implantation of a prosthetic heart valve.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (e.g., stents) and artificial valves, as well as a number of known methods of implanting these devices and valves in humans. Percutaneous and minimally-invasive surgical approaches, such as transcatheter aortic valve replacement (TAVR), are used in various procedures to deliver prosthetic medical devices to locations inside the body that are not readily accessible by surgery or where access without surgery is desirable.

As surgical approaches for valve replacement become available for younger patients, patient lifetime may exceed the corresponding lifetime of the implanted prosthetic valve. Valve-in-valve (ViV) procedures have been developed to mount a new prosthetic valve within the previously-implanted prosthetic valve. However, such procedures may pose a risk of coronary artery obstruction. In particular, the leaflets of the previously-implanted prosthetic valve may block the coronary artery ostia or otherwise inhibit blood flow through the frame of the new prosthetic valve to the coronary artery ostia. A similar problem may occur when a prosthetic valve is percutaneously expanded within a native heart valve, for example, when the native leaflets are displaced outward toward the coronary ostia. Existing methods, which rely on lacerating existing leaflets, require high spatial precision and surgical skill. Moreover, portions of the lacerated leaflet may still act to partially or completely obstruct the coronary ostia.

### SUMMARY

Described herein are embodiments of methods and tools for folding and/or capturing leaflets of a heart valve to avoid, or at least reduce the risk of, obstruction of the coronary ostia. In some embodiments, a part of a leaflet (or parts of leaflets) is captured and held distal to the coronary ostia by one or more external features of a prosthetic heart valve during installation thereof within an existing valvular structure (e.g., native heart valve or a previously implanted prosthetic heart valve). In some embodiments, a part of a leaflet (or parts of leaflets) is folded upon itself and/or held distal to the coronary ostia by one or more sutures, coupling members, locking members, clip members, and/or spiked grasping members. A prosthetic heart valve can subsequently be installed within the existing valvular structure. The captured and/or folded leaflet parts allow blood to flow to the coronary artery, the ostia of which might otherwise have been blocked by the unmodified leaflet. In other embodiments, a part of a leaflet (or parts of leaflets) is captured or folded prior to or during installation of a prosthetic heart valve within an existing valvular structure (e.g., native heart valve or a previously implanted prosthetic heart valve) at a valve position other than the aortic position (e.g., pulmonary, tricuspid, or mitral valves).

Any of the various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosed technology will become more apparent from the following detailed description, which proceeds with reference to the accompanying figures.

### BRIEF DESCRIPTION OF THE DRAWINGS

FIG. 1 shows a cross-sectional view of a native aortic valve.

FIG. 2A shows a side view of a prosthetic heart valve implanted in the native aortic valve annulus.

FIG. 2B shows the implanted prosthetic heart valve of FIG. 2A as viewed from the ascending aorta.

FIG. 2C shows an exemplary prosthetic valve delivery apparatus that can be used for implanting a prosthetic heart valve, according to one or more embodiments of the disclosed subject matter.

FIGS. 3A-3C illustrate simplified side views during prosthetic valve positioning, expansion, and mounting stages, respectively, for leaflet capture according to a first example.

FIGS. 4A-4D illustrate simplified side views of catheter positioning, leaflet piercing, suture loop formation, and tightening stages, respectively, for leaflet capture according to a second example.

FIG. 4E illustrates a simplified side view of a tightening stage using a sliding member, according to a variation of the second example.

FIG. 4F illustrates a simplified side view of a suture loop locking stage for leaflet capture according to the second example.

FIG. 4G illustrates a simplified side view of a prosthetic heart valve being implanted between the leaflet of the native aortic valve after one or more of the native leaflets have been modified.

FIG. 5A-5H illustrate simplified side views of first catheter positioning, distal-side first anchor formation, proximal-side first anchor formation, second catheter positioning, distal-side second anchor formation, proximal-side second anchor formation, coupling member sliding, and locking stages, respectively, for leaflet capture according to a third example.

FIGS. 6A-6B illustrate a capture device with actuator in an open configuration and in a closed configuration, respectively, according to a fourth example.

FIGS. 6C-6E illustrate simplified side views of capture device positioning, leaflet gathering, and locking stages, respectively, for leaflet capture according to the fourth example.

FIG. 7A illustrates a capture device according to a fifth example.

FIGS. 7B-7D illustrate simplified side views of catheter positioning, capture device partial deployment, and capture device full deployment stages, respectively, for leaflet capture according to the fifth example.

FIG. 8A illustrates a tool for leaflet folding according to a sixth example.

FIG. 8B is a simplified detailed view of the leaflet folding tool of FIG. 8A employing a tip configuration with a spike.

FIGS. 8C-8D are simplified detailed views (side and bottom, respectively) of the leaflet folding tool of FIG. 8A employing a tip configuration with a barbed spike.

FIG. 9A-9F illustrates simplified side views of leaflet folding tool positioning, leaflet engagement, distal positioning of a single leaflet, distal positioning of multiple leaflets, prosthetic heart valve positioning, and prosthetic heart valve expansion, respectively, for leaflet folding according to the sixth example.

FIG. 10A-10B illustrate a deformable capture device in an initial configuration and in a deformed configuration, respectively, according to a seventh example.

FIG. 10C illustrate the deformable capture device with actuator in a closed configuration according to the seventh example.

FIGS. 11A-11E illustrate simplified side views of capture device positioning, leaflet engagement, leaflet gathering, device deformation, and final locked stages, respectively, for leaflet capture according to the seventh example.

### DETAILED DESCRIPTION

### General Considerations

For purposes of this description, certain aspects, advantages, and novel features of the embodiments of this disclosure are described herein. The disclosed methods, apparatus, and systems should not be construed as being limiting in any way. Instead, the present disclosure is directed toward all novel and nonobvious features and aspects of the various disclosed embodiments, alone and in various combinations and sub-combinations with one another. The methods, apparatus, and systems are not limited to any specific aspect or feature or combination thereof, nor do the disclosed embodiments require that any one or more specific advantages be present, or problems be solved. The technologies from any example can be combined with the technologies described in any one or more of the other examples.

Although the operations of some of the disclosed embodiments are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used herein with reference to the prosthetic heart valve assembly and implantation and structures of the prosthetic heart valve, “proximal” refers to a position, direction, or portion of a component that is closer to the user and a handle of the delivery system or apparatus that is outside the patient, while “distal” refers to a position, direction, or portion of a component that is further away from the user and the handle, and closer to the implantation site. The terms “longitudinal” and “axial” refer to an axis extending in the proximal and distal directions, unless otherwise expressly defined.

The terms “axial direction,” “radial direction,” and “circumferential direction” have been used herein to describe the arrangement and assembly of components relative to the geometry of the frame of the prosthetic heart valve. Such terms have been used for convenient description, but the disclosed embodiments are not strictly limited to the description. In particular, where a component or action is described relative to a particular direction, directions parallel to the specified direction as well as minor deviations therefrom are included. Thus, a description of a component extending along an axial direction of the frame does not require the component to be aligned with a center of the frame; rather, the component can extend substantially along a direction parallel to a central axis of the frame.

As used herein, the terms “integrally formed” and “unitary construction” refer to a construction that does not include any welds, fasteners, or other means for securing separately formed pieces of material to each other.

As used herein, operations that occur “simultaneously” or “concurrently” occur generally at the same time as one another, although delays in the occurrence of operation relative to the other due to, for example, spacing between components, are expressly within the scope of the above terms, absent specific contrary language.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language. As used herein, “and/or” means “and” or “or,” as well as “and” and “or.”

Directions and other relative references may be used to facilitate discussion of the drawings and principles herein, but are not intended to be limiting. For example, certain terms may be used such as “inner,” “outer,” “upper,” “lower,” “inside,” “outside,”, “top,” “bottom,” “interior,” “exterior,” “left,” “right,” and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an “upper” part can become a “lower” part simply by turning the object over. Nevertheless, it is still the same part and the object remains the same.

The disclosure of numerical ranges should be understood as referring to each discrete point within the range, inclusive of endpoints, unless otherwise noted. Unless otherwise indicated, all numbers expressing quantities of components, molecular weights, percentages, temperatures, times, and so forth, as used in the specification or claims are to be understood as being modified by the term “about.” Accordingly, unless otherwise implicitly or explicitly indicated, or unless the context is properly understood by a person of ordinary skill in the art to have a more definitive construction, the numerical parameters set forth are approximations that may depend on the desired properties sought and/or limits of detection under standard test conditions/methods, as known to those of ordinary skill in the art. When directly and explicitly distinguishing embodiments from discussed prior art, the embodiment numbers are not approximates unless the word “about” is recited. Whenever “substantially,” “approximately,” “about,” or similar language is explicitly used in combination with a specific value, variations up to and including 10% of that value are intended, unless explicitly stated otherwise.

### Overview of the Disclosed Technology

Described herein are methods and tools for folding and/or capturing leaflets of a heart valve (e.g., a native heart valve or a previously implanted prosthetic heart valve). In some embodiments, the heart valve is at the aortic position and the folding/capturing is effective to avoid, or at least reduce the risk of, obstructing blood flow to the coronary arteries. In some embodiments, a part of a leaflet (or parts of leaflets) is captured and held distal to (upstream of) the coronary ostia by one or more external features of a prosthetic heart valve during installation thereof within an existing valvular structure (e.g., native heart valve or a previously implanted prosthetic heart valve). Alternatively or additionally, a part of a leaflet (or parts of leaflets) is folded upon itself and held distal to (upstream of) the coronary ostia by one or more sutures, coupling members, and/or capture devices (e.g., locking device, clip member, lowering bar, grasping member). When a new prosthetic heart valve is subsequently installed within the existing valvular structure, the captured and/or folded leaflets of the existing valvular structure are disposed at locations distal to the coronary artery ostia, thereby allowing blood to flow unobstructed to the coronary arteries. In other embodiments, the heart valve is at a position other than the aortic position, e.g., the pulmonary, tricuspid, or mitral positions.

### Examples of the Disclosed Technology

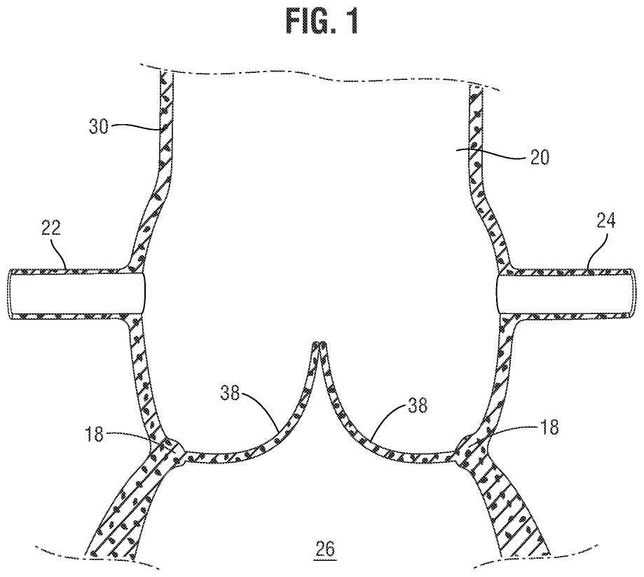

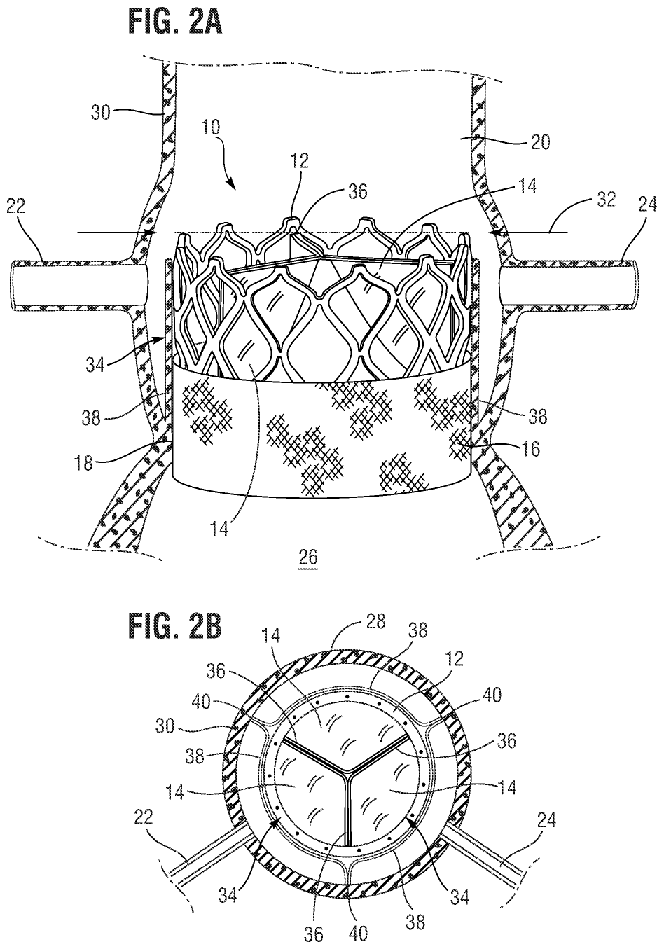

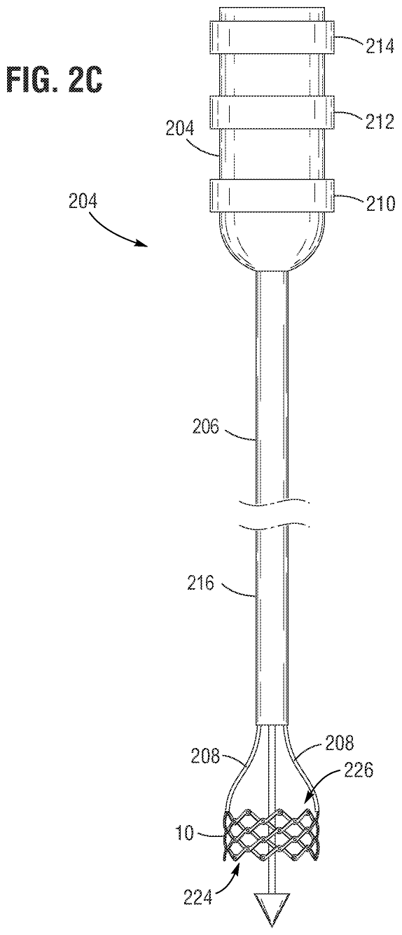

FIG. 1 shows the anatomy of the aortic root of a native valvular structure, which has a plurality of leaflets 38 (e.g., three leaflets, although only two are illustrated in the simplified illustration of FIG. 1) separating the left ventricle 26 from the ascending aorta 20. FIGS. 2A-2B show an exemplary prosthetic heart valve 10 implanted within the aortic annulus 18 of the native valvular structure. The prosthetic heart valve 10 can be radially compressible/expandable between a compressed configuration for delivery into a patient and an expanded configuration for mounting (e.g., as shown in FIG. 2A).

The prosthetic heart valve 10 can include an annular stent or frame 12. The frame 12, or components thereof (e.g., struts and/or fasteners), can be made of any of various suitable plastically-expandable materials (e.g., stainless steel, etc.) or self-expanding materials (e.g., nickel titanium alloy (NiTi), such as nitinol), as known in the art. Suitable plastically-expandable materials that can be used to form the frame 12 include, without limitation, stainless steel, a biocompatible, high-strength alloys (e.g., a cobalt-chromium or a nickel-cobalt-chromium alloys), polymers, or combinations thereof. In particular embodiments, frame 12 is made of a nickel-cobalt-chromium-molybdenum alloy, such as MP35N® alloy (SPS Technologies, Jenkintown, Pa.), which is equivalent to UNS R30035 alloy (covered by ASTM F562-02). MP35N® alloy/UNS R30035 alloy comprises 35% nickel, 35% cobalt, 20% chromium, and 10% molybdenum, by weight.

When constructed of a plastically-expandable material, the frame 12 (and thus the prosthetic valve 10) can be crimped to a radially collapsed configuration on a delivery catheter and then expanded inside a patient by an inflatable balloon or equivalent expansion mechanism. When constructed of a self-expandable material, the frame 12 (and thus the prosthetic valve 10) can be crimped to a radially collapsed configuration and restrained in the collapsed configuration by insertion into a sheath or equivalent mechanism of a delivery catheter. Once inside the body, the prosthetic valve can be advanced from the delivery sheath, which allows the prosthetic valve to expand to its functional size.

FIG. 2C illustrates an exemplary delivery apparatus 200 adapted to deliver a prosthetic heart valve, such as prosthetic heart valve 10 described herein or any other prosthetic heart valve. The prosthetic valve 10 can be releasably coupled to the delivery apparatus 200, such as via a removable coupling between a distal member of an expansion and locking mechanism of the prosthetic valve 10 and a second actuation member of an actuation assembly of the delivery apparatus 200. The prosthetic valve 10 can include a distal end 224 and a proximal end 226, wherein the proximal end 226 is positioned closer to a handle 204 of the delivery apparatus 200 than the distal end 224, and wherein the distal end 224 is positioned farther from the handle 204 than the proximal end 226. It should be understood that the delivery apparatus 200 can be used to implant prosthetic devices other than prosthetic valves, such as stents or grafts.

The delivery apparatus 200 in the illustrated example generally includes the handle 204, a first elongated shaft 206 (which comprises an outer shaft in the illustrated embodiment) extending distally from the handle 204, at least one actuator assembly 208 extending distally through the outer shaft 206. In some examples, a distal end portion 216 of the shaft 206 can be sized to house the prosthetic valve in its radially compressed, delivery state during delivery of the prosthetic valve through the patient's vasculature. In this manner, the distal end portion 216 functions as a delivery sheath or capsule for the prosthetic valve during delivery.

The at least one actuator assembly 208 can be configured to radially expand and/or radially collapse the prosthetic valve 10 when actuated, and may be removably coupled to the prosthetic heart valve 10. Although the illustrated example shows two actuator assemblies 208 for purposes of illustration, it should be understood that one actuator 208 can be provided for each actuator of the prosthetic valve. For example, three actuator assemblies 208 can be provided for a prosthetic valve having three actuators. In other examples, a greater or fewer number of actuator assemblies can be present. The actuator assemblies 208 can be releasably coupled to the prosthetic valve 10. For example, each actuator assembly 208 can be coupled to a respective actuator of the prosthetic valve 10. Each actuator assembly 208 can comprise a support tube or sleeve and an actuator member. In some examples, the actuator assembly 208 also can include a locking tool. When actuated, the actuator assembly can transmit pushing and/or pulling forces to portions of the prosthetic valve to radially expand and collapse the prosthetic valve. The actuator assemblies 208 can be at least partially disposed radially within, and extend axially through, one or more lumens of the outer shaft 206. For example, the actuator assemblies 208 can extend through a central lumen of the shaft 206 or through separate respective lumens formed in the shaft 206.

The handle 204 of the delivery apparatus 200 can include one or more control mechanisms (e.g., knobs or other actuating mechanisms) for controlling different components of the delivery apparatus 200 in order to expand and/or deploy the prosthetic valve 10. For example, in FIG. 2C, the handle 204 comprises first, second, and third knobs 210, 212, and 214. The first knob 210 can be a rotatable knob configured to produce axial movement of the outer shaft 206 relative to the prosthetic valve 10 in the distal and/or proximal directions in order to deploy the prosthetic valve from the delivery sheath 216 once the prosthetic valve has been advanced to a location at or adjacent the desired implantation location with the patient's body. For example, rotation of the first knob 210 in a first direction (e.g., clockwise) can retract the sheath 216 proximally relative to the prosthetic valve 10 and rotation of the first knob 210 in a second direction (e.g., counter-clockwise) can advance the sheath 216 distally. In other examples, the first knob 210 can be actuated by sliding or moving the knob 210 axially, such as pulling and/or pushing the knob. In other example, actuation of the first knob 210 (rotation or sliding movement of the knob 210) can produce axial movement of the actuator assemblies 208 (and therefore the prosthetic valve 10) relative to the delivery sheath 216 to advance the prosthetic valve distally from the sheath 216.

The second knob 212 can be a rotatable knob configured to produce radial expansion and/or contraction of the prosthetic valve 10. For example, rotation of the second knob 212 can move the actuator member and the support tube axially relative to one another. Rotation of the second knob 212 in a first direction (e.g., clockwise) can radially expand the prosthetic valve 10 and rotation of the second knob 212 in a second direction (e.g., counter-clockwise) can radially collapse the prosthetic valve 10. In other example, the second knob 212 can be actuated by sliding or moving the knob 212 axially, such as pulling and/or pushing the knob.

The third knob 214 can be a rotatable knob configured to retain the prosthetic heart valve 10 in its expanded configuration. For example, the third knob 214 can be operatively connected to a proximal end portion of the locking tool of each actuator assembly 208. Rotation of the third knob in a first direction (e.g., clockwise) can rotate each locking tool to advance the locking nuts to their distal positions to resist radial compression of the frame of the prosthetic valve. Rotation of the knob 214 in the opposite direction (e.g., counterclockwise) can rotate each locking tool in the opposite direction to decouple each locking tool from the prosthetic valve 10. In other embodiments, the third knob 214 can be actuated by sliding or moving the third knob 214 axially, such as pulling and/or pushing the knob.

Although not shown, in some examples, the handle 204 can include a fourth rotatable knob operative connected to a proximal end portion of each actuator member. The fourth knob can be configured to rotate each actuator member, upon rotation of the knob, to unscrew each actuator member from the proximal portion of a respective actuator. Once the locking tools and the actuator members are uncoupled from the prosthetic valve 10, they can be removed from the patient. Further details regarding construction and operation of a delivery apparatus for delivering and implanting a prosthetic heart valve can be found in U.S. Pat. Nos. 8,652,202, 9,339,384, 9,827,093, 9,867,700, 10,076,638, and 10,806,573, all of which are incorporated herein by reference.

In some embodiments, struts of the frame 12 are pivotable or bendable relative to each other to permit radial expansion and contraction of the frame 12. For example, the frame 12 can be formed (e.g., via laser cutting, electroforming or physical vapor deposition) from a single piece of material (e.g., a metal tube). In other embodiments, the frame 12 can be constructed by forming individual components (e.g., the struts and fasteners of the frame) and then mechanically assembling and connecting the individual components together.

Further details regarding the construction of the frame 12 and the prosthetic heart valve 10 are described in U.S. Patent Application Publication Nos. 2012/0123529, 2018/0153689, 2018/0344456, 2019/0060057, 2019/0365530, 2020/0188099, and 2020/0390547, and International Application Publication Nos. WO-2020/081893 and WO-2021/003167, all of which are incorporated herein by reference.

The frame 12 can have a first axial end and a second axial end. In the depicted embodiment, the first axial end (e.g., facing the ascending aorta 20 near sinotubular junction level 32) can be an outflow end, and the second axial end (e.g., facing the left ventricle 26 near aortic annulus 18) can be an inflow end. In some embodiments, the outflow end can be coupled to a delivery apparatus for delivering the prosthetic valve to the implantation site. Alternatively, the prosthetic valve 10 can be radially crimped on an inflatable balloon of a delivery apparatus for delivery to the implantation site. Implanting the prosthetic heart valve 10 within the native aortic valve can be via a transfemoral, retrograde delivery approach. Thus, in the delivery configuration of the prosthetic heart valve, the outflow end is the proximal-most end of the prosthetic valve. In other embodiments, the inflow end can be the proximal-most end of the prosthetic heart valve in the delivery configuration, depending on the particular native valve being replaced and the delivery technique that is used (e.g., transseptal, transapical, etc.). In some cases, the inflow end can be coupled to the delivery apparatus in the delivery configuration.

The prosthetic valve 10 also includes a valvular structure configured for allowing blood flow through the frame 12 in one direction. The valvular structure can be configured to regulate the flow of blood through the prosthetic heart valve 10 from the inflow end to the outflow end. The valvular structure can include, for example, a leaflet assembly formed by one or more leaflets 14 (three leaflets illustrated in FIGS. 2A-2B) made of a flexible material. Adjacent leaflets 14 can be arranged together to form commissures 36 that are coupled (directly or indirectly) to respective portions of the frame 12, thereby securing at least a portion of the leaflet assembly to the frame 12. The leaflets 14 can be made from, in whole or part, biological material, bio-compatible synthetic materials, or other such materials. Suitable biological material can include, for example, bovine pericardium (or pericardium from other sources). Further details regarding transcatheter prosthetic heart valves, including the manner in which the valvular structure can be coupled to the frame 12 of the prosthetic heart valve 10, can be found, for example, in U.S. Pat. Nos. 6,730,118, 7,393,360, 7,510,575, 7,993,394, and 8,652,202, and U.S. Patent Application Publication Nos. 2012/0123529, 2018/0325665, and 2019/0365530, all of which are incorporated herein by reference in their entireties.

The prosthetic heart valve 10 can also include one or more skirts or sealing members. For example, the prosthetic heart valve 10 can include an inner skirt mounted on the inner surface (not shown in FIGS. 2A-2B) of the frame 12 and/or an outer skirt 16 mounted on the outer surface of the frame 12. The inner skirt can be a circumferential inner skirt that spans an entire circumference of the inner surface of the frame 12. The inner skirt can function as a sealing member to prevent or decrease perivalvular leakage (e.g., when the valve is placed at the implantation site) and as an attachment surface to anchor a portion of the leaflets 14 to the frame 12. The outer skirt 16 can function as a sealing member by sealing against the tissue of the native valve annulus 18 and helping to reduce paravalvular leakage past the prosthetic valve 10. The inner and outer skirts can be formed from any of various suitable biocompatible materials, including any of various synthetic materials (e.g., polyethylene terephthalate (PET)) or natural tissue (e.g., pericardial tissue). The inner and outer skirts can be mounted to the frame using sutures, an adhesive, welding, and/or other means for attaching the skirts to the frame. Further details regarding the inner and outer skirts and techniques for assembling the leaflets to the inner skirt and assembling the skirts on the frame are disclosed in U.S. Patent Application Publication Nos. 2012/0123529, 2019/0192296, and 2019/0365530, and International Application Publication Nos. WO-2020/159783 and WO-2020/198273, all of which are incorporated herein by reference.

For an existing implanted prosthetic valve, the valvular structure may naturally degrade over time thereby requiring repair or replacement in order to maintain adequate heart functions. In a Valve-in-Valve (ViV) procedure, a new prosthetic heart valve is mounted within the existing, degrading prosthetic heart valve in order to restore proper function. However, ViV procedures may pose an increased risk of obstruction of the coronary arteries 22, 24. In particular, the mounting of the new prosthetic heart valve within the valvular structure of the existing prosthetic heart valve can displace the leaflets of the existing heart valve outwards, thereby obstructing the ostia of the coronary arteries 22, 24. Moreover, since the leaflets of the existing heart valve are disposed outside the frame of the new prosthetic valve, they may cover external surfaces of the frame, thereby creating a substantially impermeable tubular structure that occludes openings 34 in frame 12. In some patient anatomies (e.g., when the outflow of the valve 10 is at the sinotubular (STJ) level 32 and the diameter of the valve 10 is similar to the STJ diameter such that the frame 12 touches or is very close to the aortic wall 30 at the STJ level 32), the leaflets of the existing valve structure may compromise the ability for future access into the coronary arteries 22, 24 or perfusion through the valve frame 12 to the coronary arteries 22, 24 during the diastole phase of the cardiac cycle. Similar problems may occur in some patient anatomies when a prosthetic heart valve 10 is percutaneously expanded within a native valve, displacing the native leaflets 38 outward toward the coronary ostia.

To avoid obstruction of blood flow to the coronary arteries 22, 24, the leaflets of the existing heart valve (whether a native aortic valve or a previously implanted prosthetic valve) can be captured and/or folded prior to or during implantation of a new prosthetic heart valve within the existing valvular structure. In some embodiments, a new prosthetic heart valve can include one or more components on an exterior of the valve frame. The components can be designed to capture and position one, some, or all of the leaflets of the existing valvular structure away from a level of the coronary arteries as the prosthetic heart valve is installed, thereby maintaining vascular access to the arteries.

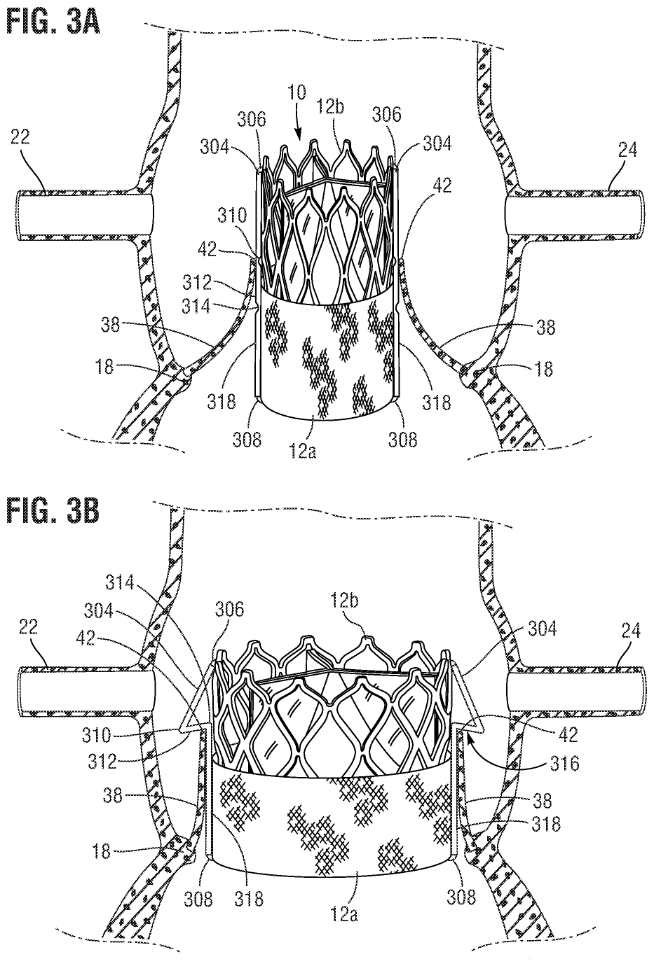

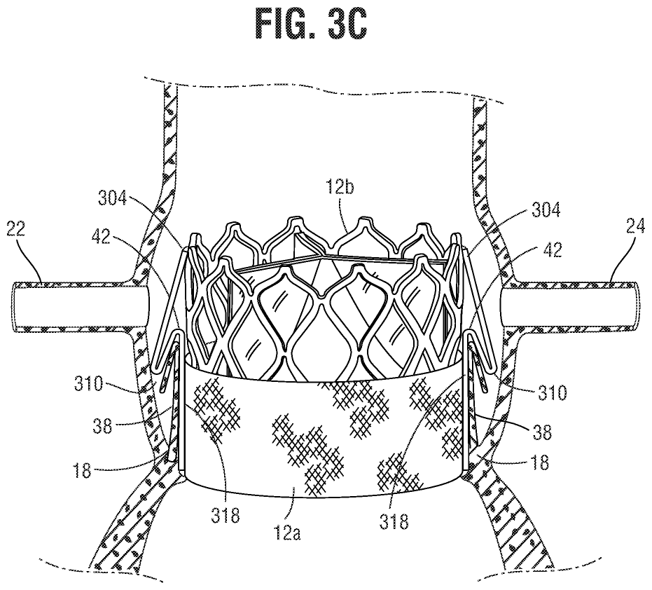

For example, FIGS. 3A-3C illustrate a prosthetic heart valve 10 that includes a leaflet capture member 304 for repositioning one or more leaflets 38 of an existing valvular structure (e.g., the native heart valve in the illustrated example, or a previously implanted prosthetic heart valve in an unillustrated example). The leaflet capture member 304 can be attached to the frame 12 of the prosthetic heart valve 10, for example, via attachment 306 at the proximal end and via attachment 308 at the distal end. For example, attachments 306, 308 can include a suture extending between the respective capture member 304 and the corresponding portion of the frame 12, a rigid attachment member (e.g., a bracket or fastener), and/or a coupling material (e.g., weld, glue, or epoxy). The leaflet capture member 304 may be otherwise free to move independent of the valve frame 12 between the attachments 306, 308.

In the description of the disclosed embodiments, the methods and devices are described in the context of using a retrograde delivery approach to the native aortic valve. As such, the term “proximal end” of the prosthetic valve (or other device) or a component thereof is used to refer to its outflow end and the term “distal end” of the prosthetic valve (or other device) or a component thereof is used to refer to its inflow end. However, it should be noted if delivered in the opposite direction to the aortic valve (e.g., transapically) the outflow end of the prosthetic valve would be the distal end and the inflow end of the prosthetic valve would be the proximal end during delivery. Thus, in the present application, once a prosthetic valve is implanted at the aortic position, the term “proximal end” is intended to mean “outflow end” and the term “distal end” is intended to mean “inflow end”. Similarly, the terms “distal side” and “distal” as used herein to describe components or parts of the anatomy are intended to mean “upstream side” and “upstream” while the terms “proximal side” and “proximal” as used herein to describe components or parts of the anatomy are intended to mean “downstream side” and “downstream.” Further, any of the methods and devices described herein can be applied to any of the native valves of the heart (the aortic, mitral, tricuspid, and pulmonary valves) or a prosthetic valve previously implanted within any of the native valves of the heart using any known techniques, which can involve approaching a native valve in a retrograde or antegrade direction.

Each leaflet capture member 304 can have a middle portion 312 connected to an upper portion by first inflection portion 310 at its proximal end and connected to a lower portion 318 by second inflection portion 314 at its distal end. For example, each inflection portion 310, 314 can comprise a weakened, notched, or grooved portion or a region with a narrowed cross-section. In some embodiments, the leaflet capture member 304 can be formed as a substantial straight bar that extends along an axial direction of the valve 10. Alternatively, the leaflet capture member 304 can have a cell-like open configuration over at least a portion thereof, for example, the portion between attachment 306 and the first inflection portion 310 so as to allow blood flow therethrough or other access to the coronary arteries 22, 24. The cell-like open configuration of the leaflet capture member 304 may have a similar configuration to and be aligned with the cell-like open configuration of the frame 12 of the prosthetic heart valve 10 (e.g., similar in size/shape to and/or aligned with opening 34 in frame 12).

In some embodiments, each leaflet capture member 304 can be disposed at a location along a circumferential direction of the valve 10 corresponding to the commissures of the valvular structure of the prosthetic valve, for example, on a side of the frame 12 directly opposite where a commissure attaches to the frame 12. In other embodiments, the leaflet capture members 304 can be disposed at other locations, for example, at locations along a circumferential direction of the valve 10 that correspond with locations of the ostia of the coronary arteries 22, 24 once the valve 10 is implanted. Although two leaflet capture members 304 are illustrated, fewer or additional members 304 are also possible, for example, a leaflet capture member 304 corresponding to each leaflet of the existing valvular structure. In addition, although illustrated as an axially-extending bar in the figures, other shapes for the leaflet capture member 304 are also possible according to one or more contemplated embodiments.

FIG. 3A shows the heart valve 10 in the initial crimped state within the existing valvular structure (e.g., the native aortic valve). The leaflet capture member 304 can have a substantially straight longitudinal configuration and can be positioned to contact a portion of the leaflets (e.g., a free end 42 of leaflet 38). As the heart valve 10 is expanded radially, the heart valve 10 contracts along its axis, thereby compressing the leaflet capture members 304 via attachments 306, 308. As attachments 306, 308 approach each other during expansion of the valve 10, axial pressure can be exerted on the leaflet capture member 304. Under application of the axial pressure, inflection portion 310 may be constructed to deflect radially outward and inflection portion 314 may be constructed to deflect radially inward, as illustrated in FIG. 3B. The folding of the leaflet capture member 304 about inflection points 310, 314 can form a pocket 316 between the middle portion 312 and the lower portion 318 of the leaflet capture member 304. In particular, the middle portion 312 can rotate about the second inflection portion 314 such that first inflection portion 310 is disposed closer to the distal end 12a of the prosthetic heart valve 10 than the second inflection portion 312. As the heart valve 10 is further expanded into its final configuration with the external wall of the valve frame 12 contacting the surrounding structure (e.g., the native annulus or the frame of the previously installed prosthetic valve), the middle portion 312 approaches the lower portion 318 such that the free end 42 of the leaflet 38 (or other portion of each leaflet) folds upon itself and is captured within pocket 316. The leaflet 38 is thus spaced from the coronary arteries 22, 24 once the heart valve 10 is fully installed, as shown in FIG. 3C.

In some embodiments, a flexible fiber (e.g., suture, wire, or thread) can be used to fold a leaflet upon itself and capture the folded leaflet in a position that avoids obstructing the corresponding coronary ostium. The flexible fiber can pierce the leaflet near a distal end thereof and can pass around the free end of the leaflet. The end of the fiber can be tied to itself with a sliding knot, thereby forming a loop containing therein part of the leaflet that is between the pierced portion and the free end. Sliding the knot in a distal direction (e.g., toward the existing heart valve) can reduce a size of the loop, which causes the leaflet part therein to curl or fold upon itself. Once sufficient compaction of the leaflet is achieved (e.g., reduction in size and/or location that would not obstruct the ostia of the coronary arteries once a prosthetic heart valve is installed within the existing valvular structure), the knot can be locked in place to retain the leaflet therein, and the new prosthetic heart valve can be subsequently installed.

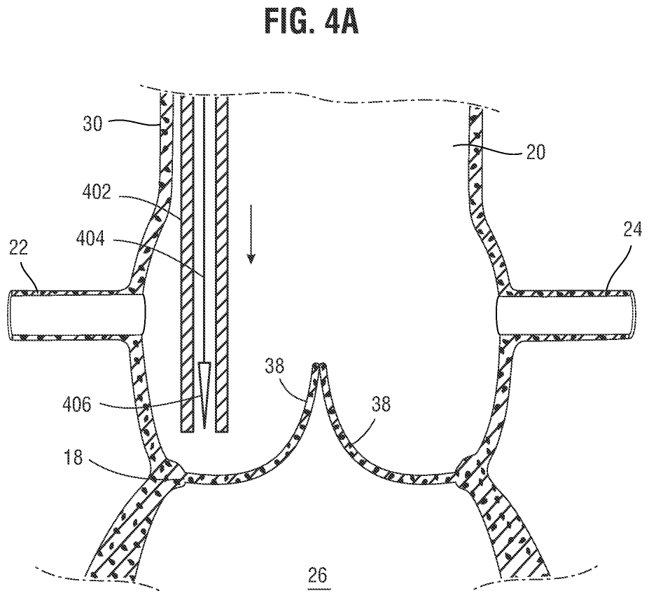

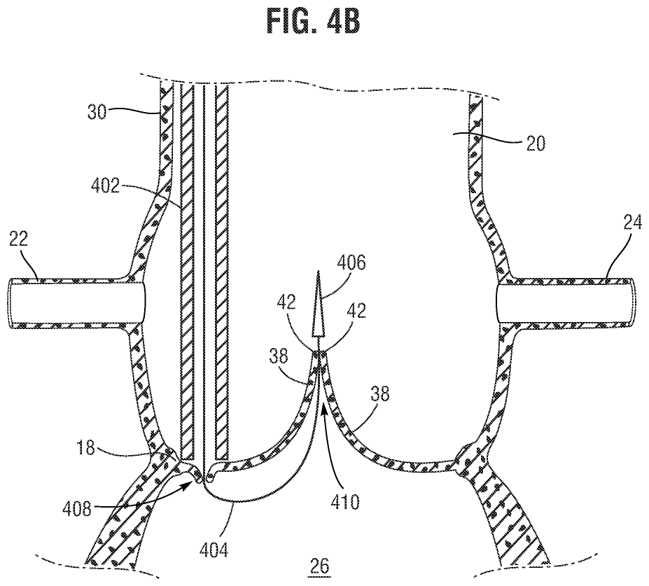

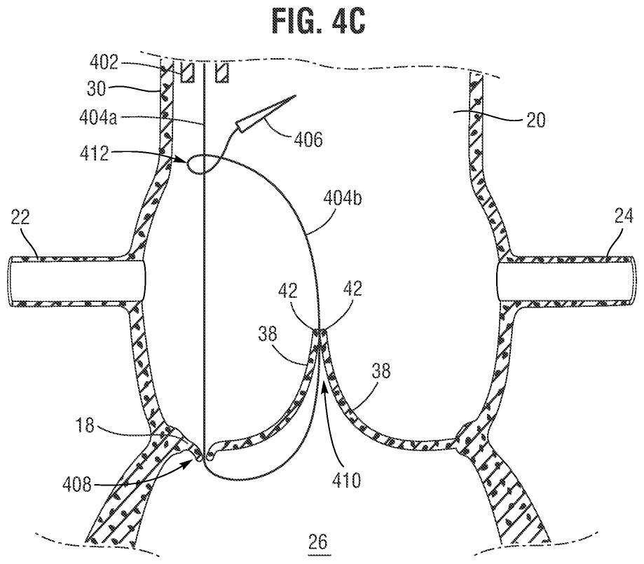

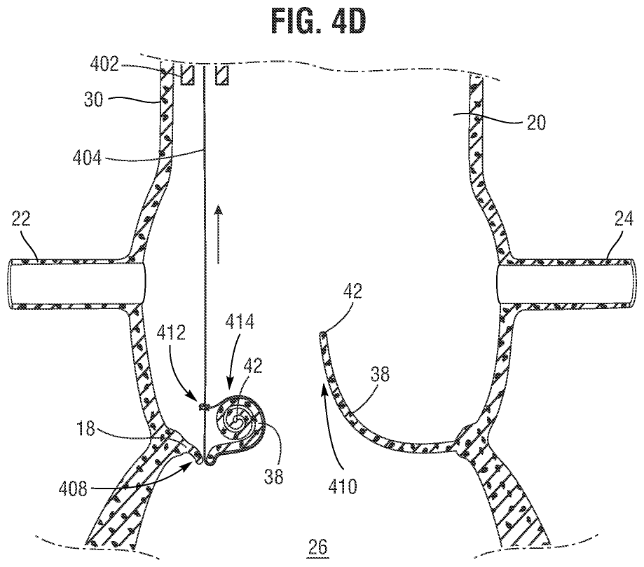

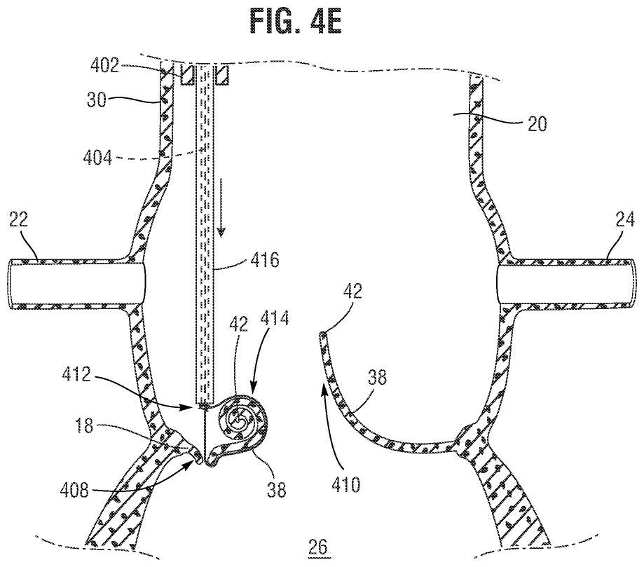

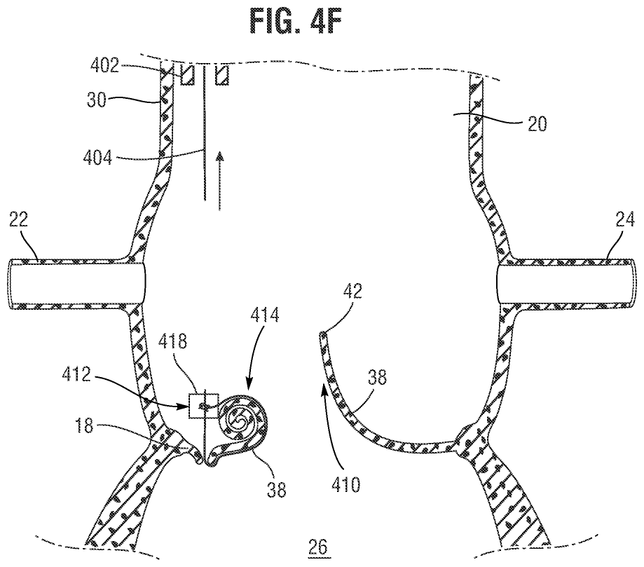

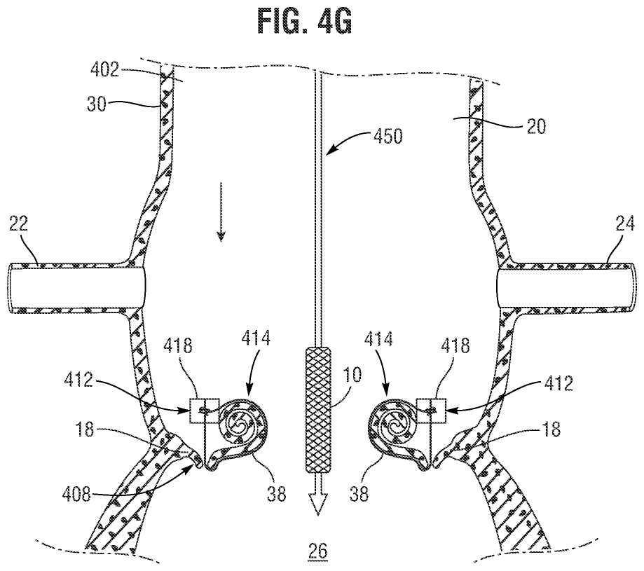

FIGS. 4A-4G illustrate an exemplary method for folding and/or capturing a leaflet using a suture. As shown in FIG. 4A, a delivery catheter 402 can be advanced from the ascending aorta 20 toward one of the leaflets 38 of an existing valvular structure (e.g., a native aortic valve in the illustrated example, or a previously installed prosthetic heart valve in an unillustrated example). For example, the delivery catheter 402 can be positioned with its end facing a first portion of one of the leaflets 38. The first portion can be closer to the valve annulus 18 than the free end 42 of the leaflet 38 and is preferably distal to the ostium of coronary artery 22. In some embodiments, the first portion of leaflet 38 may be circumferentially aligned with the ostium of coronary artery 22 (e.g., aligned or substantially aligned along a same radial vector extending from a center of the existing valvular structure, as viewed from the ascending aorta 20).

A needle 406 with a suture 404 attached thereto can be disposed within the delivery catheter 402. Once the delivery catheter 402 is disposed adjacent to the first portion of the leaflet 38, the needle 406 can be advanced out of the distal end of the delivery catheter 402 to pierce the first portion 408 of the leaflet 38. The needle 406 with suture 404 thus moves through the first portion 408 to a distal side of the leaflet 38, as shown in FIG. 4B. The suture 404 can then be pulled back to the proximal side of the leaflet 38 by moving the needle 406 through central gap 410 between free ends 42 of the leaflets 38 of the existing valvular structure. A portion 404b of suture 404 that has passed through the central gap 410 can then be tied over a portion 404a of suture 404 that has not passed through the first portion 408 of the leaflet 38.

A knot 412 is formed by the tying of portion 404b to portion 404a, as shown in FIG. 4C. Knot 412 and suture portions 404a, 404b define a loop 414 that surrounds part of the leaflet 38 between the first portion 408 and leaflet free end 42. The knot 412 can be formed so as to be slidable along the suture 404. In some embodiments, the configuration of the knot 412 itself allows it to be slidable along the suture 404, for example, by forming a slip knot or other sliding knot. Alternatively or additionally, the knot 412 may be initially formed in a loose state to allow the knot 412 to slide along the suture 404. The knot 412 can thus be slid distally along the suture 404 toward the valvular structure so as to reduce a size of the loop 414. The reduction in the size of the loop 414 causes the part of the leaflet contained therein (i.e., the part of the leaflet 38 between its free end 42 and the pierced first portion 408) to curl or fold upon itself, as shown in FIG. 4D.

In some embodiments, the sliding of the knot 412 distally toward the valvular structure can be effected by moving at least one of the knot 412 and the suture 404 with respect to the other. For example, the suture 404 can be pulled in the proximal direction, which can tighten the loop 414 and cause the knot 412 to move distally. Alternatively or additionally, the knot 412 can be pushed in the distal direction toward the pierced first portion 408 of the leaflet 38 by a push/assist member, for example, by sliding member 416 as shown in FIG. 4E. Alternatively or additionally, the knot 412 can be held in place by the push/assist member (e.g., a positioning member) as the suture 404 is retracted in the proximal direction. The sliding member 416 can have a through-hole, conduit, or recess through which suture 404 extends and can have a portion (e.g., distal end) constructed to abut the knot 412 when the sliding member 416 is brought in contact therewith. For example, the sliding member 416 can be initially disposed on the suture 404 within the delivery catheter 402, and the sliding member 416 can be sized and shaped to be delivered into the ascending aorta 20 from a distal end of the delivery catheter 402. Alternatively, the push/assist member can be a distal end portion of the delivery catheter 402 rather than a separate member 416.

The amount of reduction in the size of the loop 414 can be chosen such that the folded part of the leaflet 38 contained therein is positioned distal to the ostium of coronary artery 22, even when pressed radially outward by subsequent mounting of a prosthetic heart valve within the existing valvular structure. Once sufficient reduction in the size of the suture loop 414 is achieved, the knot 412 can be locked in place, as shown in FIG. 4F. The locking of the knot 412 can ensure that the captured part of the leaflet 38 does not unfold during or after installation of the prosthetic heart valve. For example, the knot 412 can be locked in place by manipulation of the knot itself, such as by tightening the knot 412 and/or bonding, fusing, or encasing the suture portions forming the knot 412. Alternatively or additionally, the knot 412 can be locked in place by a separate physical device 418 that is brought into contact with the knot 412. In some embodiments the sliding member 416 can be used to position the locking device 418 in place with respect to knot 412. The locking device 418 may be constructed to reconfigure between a sliding configuration (e.g., where a through-hole allows relative motion between suture 404 and locking device 418) and a non-sliding configuration (e.g., where the through-hole restricts relative motion between suture 404 and locking device 418, for example, by a member that presses suture 404 into contact with a sidewall of the through-hole). Alternatively or additionally, locking device 418 comprises a suture clip or fastener, for example, as described in U.S. Patent Application Publication Nos. 2018/0177503 and 2020/0000458, all of which are incorporated herein by reference. With knot 412 locked in place, suture 404 can be cut (e.g., at a location immediately adjacent to the locking device 418 or at another location proximal to a location of the knot 412) and retracted into delivery catheter 402, as shown in FIG. 4F. The cutting of the suture 404 can be made by a cutting tool provided from a distal end of delivery catheter 402 or provided via a separate catheter in the ascending aorta 20. Alternatively, part of delivery catheter 402 (e.g., a distal end of the catheter 402) can include a cutting element for cutting the suture 404.

The suture loop 414 thus folds part of leaflet 38 and captures it distal to coronary artery 22, thereby preventing, or at least reducing the risk of obstruction of the ostium of coronary artery 22. If additional capture/folding of one or more leaflets 38 of the existing valvular structure is desired, for example, to prevent, or at least reduce the risk of, obstruction of the ostium of coronary artery 24, then the techniques of FIGS. 4A-4F can be repeated in a similar manner with respect to the next leaflet 38 using the same delivery catheter 402 or a different delivery catheter. In such repetition, the portion of the next leaflet 38, which is pierced by the needle 406 and suture 404, can be circumferentially aligned with the ostium of coronary artery 24 (e.g., aligned or substantially aligned along a same radial vector extending from a center of the existing valvular structure, as viewed from the ascending aorta 20). Although not discussed in detail above, manipulation of the needle 406 and/or suture 404 within the patient's anatomy, movement of the push/assist member (e.g., sliding member 416) or locking device 418, and/or manipulation of the knot 412 (e.g., to move or lock of the knot) may be performed using any tools employed in laparoscopic and/or transcatheter heart surgeries, such as, but not limited to, knot pushers, suture cutters, manipulators, and the like.

When no further leaflet capture is desired, a new prosthetic heart valve in a crimped state can subsequently be advanced to the existing valvular structure with captured leaflets 38. For example, as depicted in FIG. 4G, a new prosthetic valve 10 (which can be the valve 10 of FIG. 2A or a different prosthetic valve) can be radially crimped on a balloon of a delivery apparatus 450. The delivery catheter 450 can be advanced through the aorta toward the native aortic valve to position the prosthetic valve 10 between the leaflets 38. The new prosthetic valve 10 is then expanded by inflating the balloon of the delivery catheter, such that the captured leaflets 38 are disposed on an external surface of the new valve frame. However, the locked suture loops 414 retain the capture leaflets 38 in a location distal to the coronary arteries 22, 24, thereby allowing blood to flow from the outflow end of the new prosthetic valve to the coronary arteries 22, 24 once the heart valve implantation is completed. Further details of the delivery apparatus and methods for implanting a prosthetic valve using the delivery apparatus are disclosed in U.S. Pat. Nos. 7,780,723, 9,061,119, and 9,339,384, and U.S. Patent Application Publication No. 2017/0065415, all of which are incorporated herein by reference.

In other embodiments, the prosthetic valve 10 can be a self-expandable prosthetic valve that is retained in a radially compressed state within a capsule or delivery sheath of a delivery apparatus, such as disclosed in U.S. Patent Application Publication No. 2014/0343670 and U.S. Pat. No. 8,652,202, all of which are incorporated herein by reference. Once the prosthetic valve is positioned between the leaflets 38, the prosthetic valve can be deployed from the delivery sheath, which allows the prosthetic valve to self-expand to a radially expanded state against the leaflets.

In other embodiments, the prosthetic valve 10 can be a mechanically expandable prosthetic valve that is releasably connected to one or more mechanical actuators of a delivery apparatus, such as disclosed in U.S. Patent Application Publication No. 2018/0153689, U.S. Application No. 62/990,299, and International Application No. PCT/US2020/063104, all of which are incorporated herein by reference. The prosthetic valve is retained in a radially compressed state by the delivery apparatus (optionally in a delivery sheath) and positioned between the leaflets 38. Once positioned, the prosthetic valve can be deployed from the delivery sheath and radially expanded to a radially expanded state against the leaflets by actuating the one or more mechanical actuators of the delivery apparatus.

In some embodiments, a first member (e.g., suture, wire, thread, or other structure) is tethered to a first portion of the leaflet, and a second member (e.g., suture, wire, thread, or other structure) is tethered to a second portion of the leaflet. The second portion of the leaflet can be near a free end of the leaflet, while the first portion of the leaflet can be near a base or anchor portion of the leaflet (e.g., a distal-most part of the leaflet). The first and second members can be tethered to the respective first and second portions by one or more anchors or plications. By pulling the first and second members toward each other, the second portion of the leaflet is pulled toward the first portion of the leaflet, thereby folding part of the leaflet between the first and second portions. Once sufficient folding of the leaflet is achieved (e.g., reduction in size and/or location that would not obstruct the ostia of the coronary arteries once a prosthetic heart valve is installed within the existing valve structure), the locations of the first and second members with respect to each other can be locked to retain the folded configuration of the leaflet, and the new prosthetic heart valve can be subsequently installed.

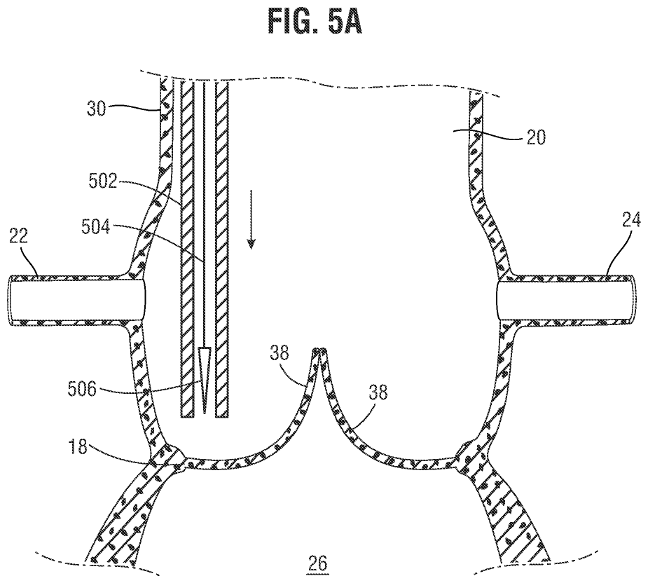

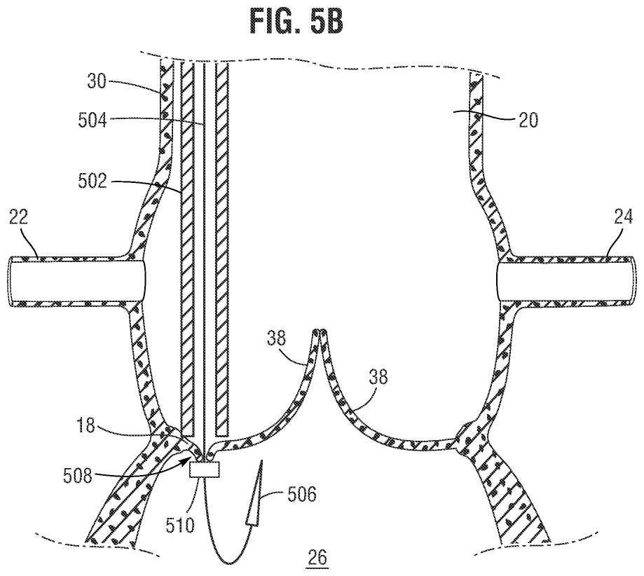

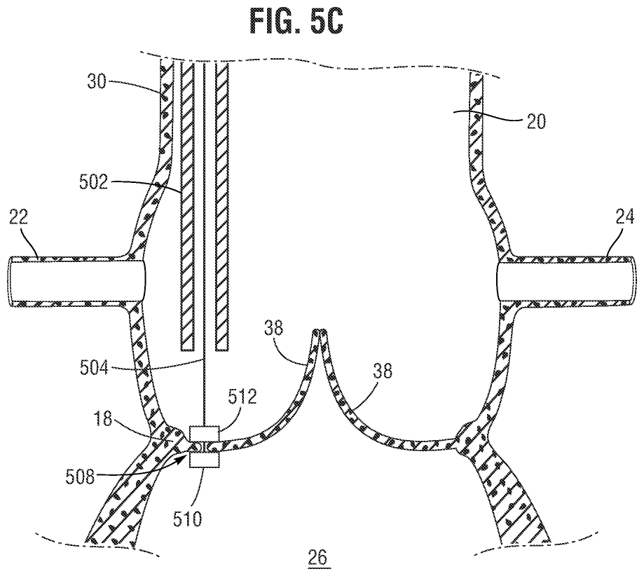

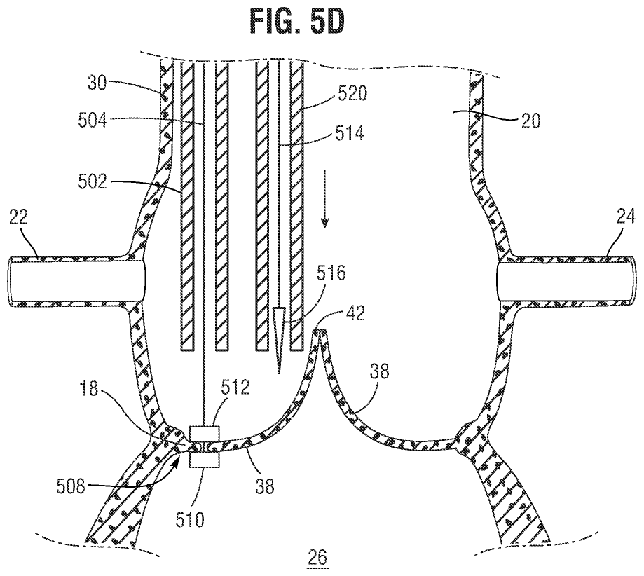

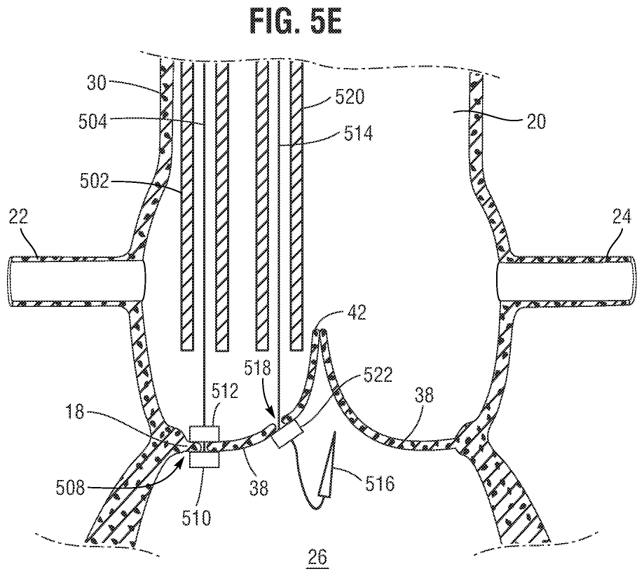

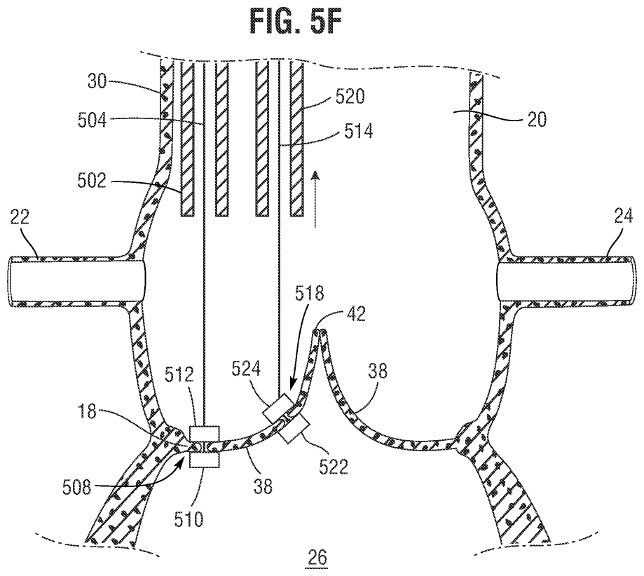

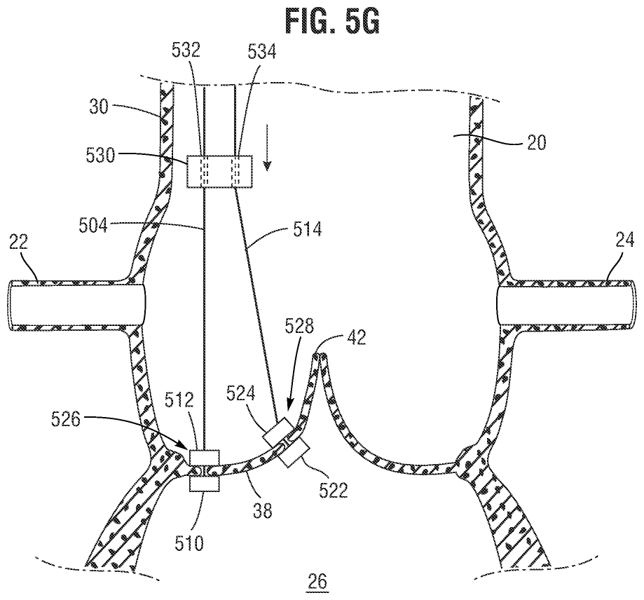

FIGS. 5A-5H illustrate an exemplary method for folding and/or capturing a leaflet using tethered sutures. As shown in FIG. 5A, a delivery catheter 502 can be advanced from the ascending aorta 20 toward one of the leaflets 38 of an existing valvular structure (e.g., a native aortic valve in the illustrated example, or a previously installed prosthetic heart valve in an unillustrated example). For example, the delivery catheter 502 can be positioned with its end facing a first portion of one of the leaflets 38. The first portion can be closer to the valve annulus 18 than the free end 42 of the leaflet 38 and is preferably distal to the ostium of coronary artery 22. In some embodiments, the first portion of leaflet 38 may be circumferentially aligned with the ostium of coronary artery 22 (e.g., aligned or substantially aligned along a same radial vector extending from a center of the existing valvular structure, as viewed from the ascending aorta 20).

A needle 506 with a first suture 504 attached thereto can be disposed within the delivery catheter 502. Once the delivery catheter 502 is disposed adjacent to the first portion of the leaflet 38, the needle 506 can be advanced out of the distal end of the delivery catheter 502 to pierce the first portion 508 of the leaflet 38. The needle 506 with first suture 504 thus moves through the first portion 508 to a distal side of the leaflet 38, as shown in FIG. 5B. At the first portion 508, at least one anchor can be formed to tether the first suture 504 to the leaflet 38. For example, a distal-side anchor 510 can be formed at first portion 508. Alternatively or additionally, a proximal-side anchor 512 can be formed at first portion 508. As shown in FIG. 5C, the first suture 504 can extend between the distal-side anchor 510 and the proximal-side anchor 512.

For example, each anchor 510, 512 can include a knotted portion of the first suture 504, a piece of cloth or fabric, a plication formed from the leaflet, and/or a locking member. To form the distal-side anchor, the anchoring components (e.g., fabric, clip, etc.) can be passed through the pierced portion of the leaflet with the suture and can be expanded on the distal-side. To form the proximal-side anchor, the anchoring components can be conveyed to the proximal-side surface of the leaflet, for example, by sliding down the suture from the delivery catheter. For example, each anchor 510, 512 can comprise one or more of the suture fasteners or suture clips disclosed in U.S. Patent Application Publication Nos. 2018/0177503 and 2020/0000458, all of which are incorporated herein by reference. Further details of suture attachment to leaflets can be found in U.S. Patent Application Publication No. 2015/0230919, which is also incorporated herein by reference.

As shown in FIG. 5D, another delivery catheter 520 can be advanced from the ascending aorta 20 and positioned with its distal end facing a second portion of the same leaflet 38. The second portion can be closer to the free end 42 of the leaflet 38 than the first portion 508. In some embodiments, the second portion of leaflet 38 may also be circumferentially aligned with the ostium of coronary artery 22 (e.g., aligned or substantially aligned along a same radial vector extending from a center of the existing valvular structure, as viewed from the ascending aorta 20). Another needle 516 with a second suture 514 attached thereto can be disposed within the delivery catheter 520. Once the delivery catheter 520 is disposed adjacent to the second portion of the leaflet 38, the needle 516 can be advanced out of the distal end of the delivery catheter 502 to pierce the second portion 518 of the leaflet 38. The needle 516 with second suture 514 thus moves through the second portion 518 to the distal side of the leaflet 38, as shown in FIG. 5E.

At the second portion 518, at least one anchor can be formed to tether the second suture 514 to the leaflet 38. For example, a distal-side anchor 522 can be formed at second portion 518. Alternatively or additionally, a proximal-side anchor 524 can be formed at second portion 518. As shown in FIG. 5F, the second suture 514 can extend between the distal-side anchor 522 and the proximal-side anchor 524. Similar to anchors 510, 512, each anchor 522, 524 can be a knotted portion of the first suture 504, a plication formed from the leaflet, and/or a locking member and/or can employ one or more of the suture fasteners or suture clips. Although the positioning of delivery catheter 520 and formation of anchors 522, 524 at the second portion 518 has been described after the positioning of delivery catheter 502 and formation of anchors 510, 512 at the first portion 508, such description has been for convenience only. Indeed, the steps described above with respect to FIGS. 5A-5F may occur at the same time or in a different order from that described.

Once sutures 504, 514 are tethered to the first portion 508 and second portion 518 of the leaflet 38 by their respective anchors, the delivery catheters 502, 520 can be retracted and a coupling member 530 can be provided, as shown in FIG. 5G, which can, for example, be delivered via another catheter inserted over the sutures 504, 514 through the ascending aorta. The coupling member 530 can have one or more through-holes, conduits, or recesses through which sutures 504, 514 extend. For example, coupling member 530 can be a sliding member with a pair of through-holes 532, 534. The first suture 504 can extend through the first through-hole 532 while the second suture 514 can extend through the second through-hole 534. A lateral distance between the through-holes 532, 534 can be less than a distance between the first portion 508 and the second portion 518 along a contour of the leaflet 38. Thus, as the coupling member 530 is advanced distally from the ascending aorta 20 toward the leaflet 38, the spacing between the through-holes 532, 534 pulls the first and second portions 508, 518 of the leaflet 38 toward each other, thereby causing the leaflet 38 to fold. In particular, the part 538 of the leaflet between anchored portion 526 (e.g., closest to valve annulus 18) and anchored portion 528 (e.g., closest to free end 42) can fold upon itself to have a corrugated configuration between anchors 510/512 and anchors 522/524, or at least to extend distally away from coronary artery 22, as illustrated in FIG. 5H. Alternatively, the coupling member can be a sliding member with a single through-hole. Both the first and second sutures 504, 514 can extend through the same through-hole. A lateral dimension (e.g., diameter) of a distal end of the through-hole can be less than a distance between the first portion 508 and the second portion 518 along the contour of the leaflet 38. Thus, as the coupling member is advanced toward the leaflet 38, the sidewalls of the through-hole pull the first and second portions 508, 518, of the leaflet 38 toward each other, thereby causing the leaflet 38 to fold.

In some embodiments, the delivery catheter 502 used to tether the first suture 504 to the first portion 508 of the leaflet 38 can be the same as that used to tether the second suture 514 to the second portion 518. For example, after forming the anchors 510, 512 at the first portion 508, the suture 504 can be cut at a location between the distal end of the delivery catheter 502 and the anchor 512, and the severed part of suture 504 retained for subsequent use with a coupling member. The delivery catheter 502 is thus free to reposition with respect to the second portion 518, e.g., as delivery catheter 520 in FIG. 5F. Alternatively, after forming the anchors 510, 512 at the first portion 508, the first suture 504 is maintained extending from the end of the delivery catheter 502 during the repositioning with respect to the second portion 518. The second needle 516 and the second suture 514 can be extended from the delivery catheter 502 while the first suture 504 continues to extend between the first portion 508 and the catheter 502. In such configurations, the delivery catheter 502 may be used as a “single through-hole” coupling member to pull the first portion 508 and the second portion 518 together after forming anchors 522, 524.

Once the coupling member 530 is in contact with the proximal-side anchors 512, 524 and/or when the leaflet has otherwise achieved sufficient folding (the folded leaflet 38 is positioned distal to the ostium of coronary artery 22, even when pressed radially outward by subsequent mounting of a prosthetic heart valve within the existing valvular structure), the coupling member 530 can be locked in place with respect to anchored portions 526, 528, as shown in FIG. 5H. The locking of the coupling member 530 can ensure that the folded part of the leaflet 38 does not unfold during or after installation of the prosthetic heart valve. For example, the coupling member 530 can be locked in place by a separate physical device 536 that is brought into contact with the proximal side of the coupling member 530. Alternatively or additionally, the coupling member 530 may be constructed to be reconfigured between a sliding configuration (e.g., where through-holes 532, 534 allow relative motion between sutures 504, 514 and the coupling member 530) and a non-sliding configuration (e.g., where each through-hole 532, 534 restricts relative motion between sutures 504, 514 and coupling member 530, for example, by a member that presses sutures 504, 514 into contact with a sidewall of the respective through-hole). Alternatively or additionally, the coupling member 530 can be locked in place by manipulation of the sutures themselves. For example, free ends of sutures 504, 514 extending out of the proximal-side of the coupling member 530 can be joined together (e.g., by tying, bonding, fusing, or encasing) in order to lock the coupling member in place.

With coupling member 530 locked in place, each suture 504, 514 can be cut (e.g., at a location immediately adjacent to the locking device 536 or at another location proximal to a location of the coupling member) and retracted proximally, as shown in FIG. 5H. The cutting of each suture 504, 514 can be made by a cutting tool provided from a distal end of either delivery catheter 502, 520 or provided via a separate catheter in the ascending aorta 20. Alternatively, part of one or both of the delivery catheters 502, 520 (e.g., distal ends of catheters 502, 520) can include a cutting element for cutting the respective suture 504, 514.

The anchored portions 526, 528 pulled toward each other thus capture and fold leaflet part 538 and position the leaflet free end 42 distal to coronary artery 22, thereby preventing, or at least reducing the risk of obstruction of the ostium of coronary artery 22. If additional capture/folding of one or more leaflets 38 of the existing valvular structure is desired, for example, to prevent, or at least reduce the risk of, obstruction of the ostium of coronary artery 24, then the techniques of FIGS. 5A-5H can be repeated in a similar manner with respect to the next leaflet 38 using the same delivery catheters 502, 520 or different delivery catheters. In such repetition, the portions of the next leaflet 38, which are pierced by needles 506 and 516 respectively, can be circumferentially aligned with the ostium of coronary artery 24 (e.g., aligned or substantially aligned along a same radial vector extending from a center of the existing valvular structure, as viewed from the ascending aorta 20). Although not discussed in detail above, manipulation of needles 506, 516 and/or sutures 504, 514 within the patient's anatomy, forming or installing anchors 510, 512, 522, 524, and/or movement of the coupling member 530 or locking device 536 may be performed using any tools employed in laparoscopic and/or transcatheter heart surgeries, such as, but not limited to, suture cutters, manipulators, and the like.

When no further leaflet capture/folding is desired, a new prosthetic heart valve in a crimped state can subsequently be advanced to the existing valvular structure with captured leaflets 38 (such as shown in FIG. 4G). The new valve is disposed within the valvular structure and expanded, such that the captured leaflets 38 are disposed on an external surface of the new valve frame. However, the proximated anchored portions 526, 528 retain the leaflet 38 in a position distal to the coronary arteries 22, 24, thereby allowing blood to flow from the outflow end of the new prosthetic valve to the coronary arteries 22, 24 once the heart valve implantation is completed.

In some embodiments, a locking device can be used to capture a leaflet in a folded configuration and at a position that avoids obstructing the corresponding coronary ostium. The locking device can be advanced in an open configuration over a free end of the leaflet. A portion of the locking device or of an actuator thereof can gather part of the leaflet between opposing members of the locking device as the locking device is further advanced over the leaflet. Once a sufficient part of the leaflet has been gathered (e.g., reduction in size and/or location that would not otherwise obstruct the ostia of the coronary arteries once a prosthetic heart valve is installed within the existing valvular structure), the locking device can transition to the closed configuration, where the locking device members approach each other to contact and retain the gathered part of the leaflet therebetween, and the new prosthetic heart valve can be subsequently installed.

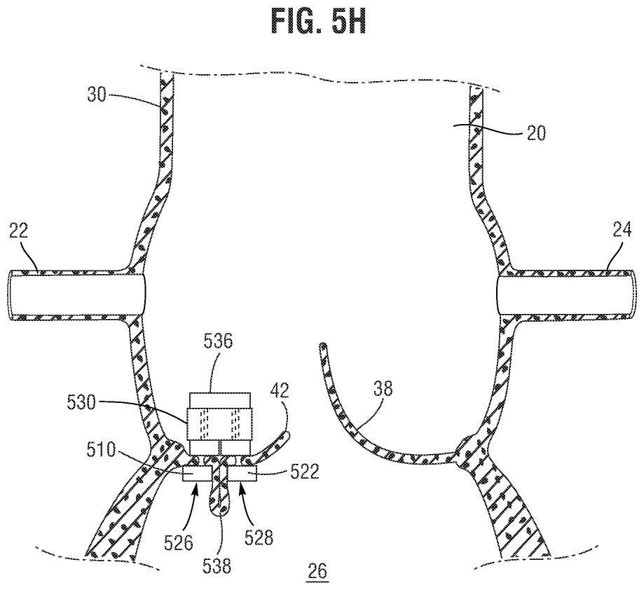

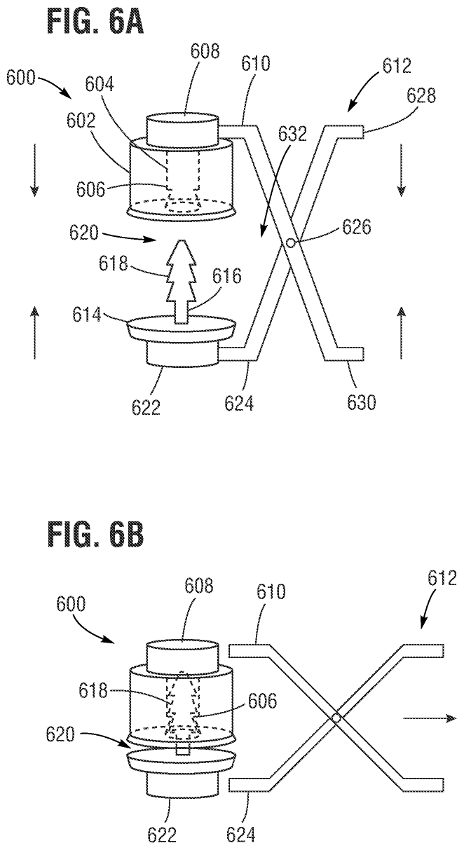

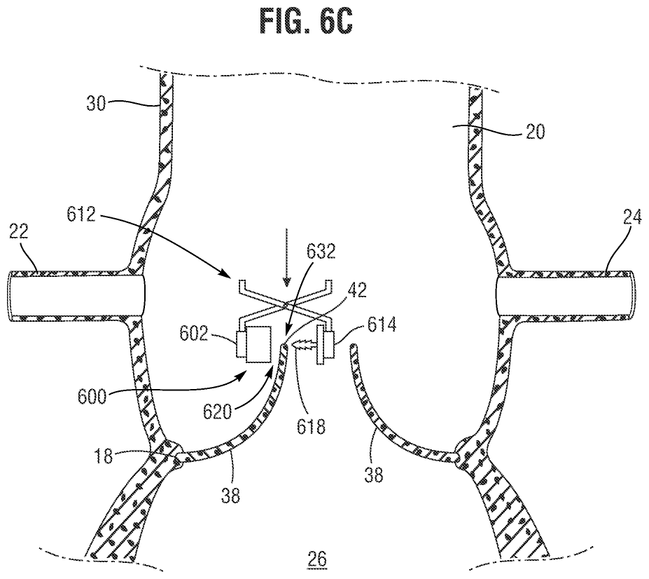

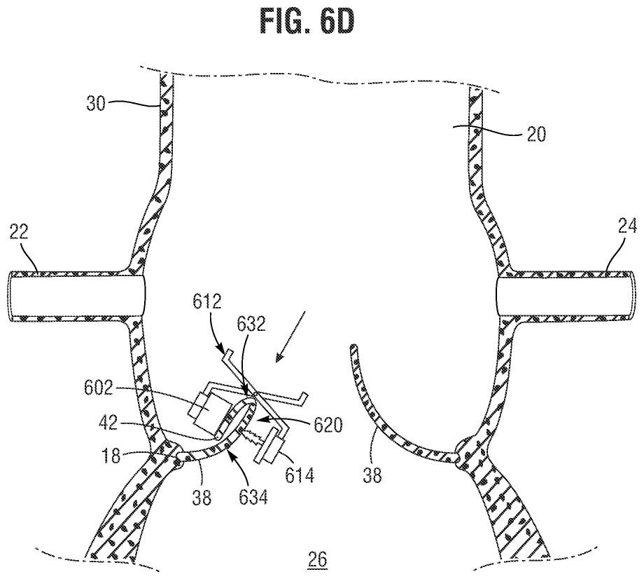

FIGS. 6A-6B illustrate an exemplary locking device, and FIGS. 6C-6E illustrate an exemplary method for folding and/or capturing a leaflet using the exemplary locking device. In particular, FIG. 6A illustrates the locking device 600 in an open configuration, and FIG. 6B illustrates the locking device 600 in the closed configuration. The locking device 600 can have a first member 602 and a second member 614. In the open configuration, the first member 602 is spaced from the second member 614 to define a gap 620 therebetween. In the closed configuration, the first member 602 and the second member 614 approach each other to eliminate, or at least reduce a size of, the gap 620. The first member 602 can be constructed as a female member, for example, having one or more recesses 604. The second member 614 can be constructed as a male member, for example, one or more projections or tines 616. Each projection 616 corresponds to one of the recesses 604, with the projection 616 being spaced from and aligned with the corresponding recess 604 in the open configuration, and the projection 616 being inserted into and in contact with a portion of the corresponding recess 604 in the closed configuration. Each projection 616 can have a sharp tip constructed to pierce a leaflet within gap 620 as the locking device 600 transitions from the open configuration to the closed configuration.

The projection 616 and the recess 604 can be constructed with one or more snap-fit features, for example, to prevent removal of projection 616 from recess 604 once the projection 616 has been inserted into the recess 604 in the closed configuration. For example, projection 616 may include a barbed portion 618, and recess 604 may include a ridge 606 therein. As the locking device 600 transitions to the closed configuration, the projection 616 is inserted into the recess 604 such that an edge of barbed portion 618 abuts ridge 606 to resist withdrawal of the projection 616 from the recess 604. In some embodiments, the projection 616 includes multiple barbed portions, for example, to accommodate a variation in thicknesses for the gathered portion of the leaflets between the first and second members 602, 614. In addition to snap-fit locking features between the recess 604 and projection 616, or in place thereof, the locking device 600 can include one or more external locking features, such as, but not limited, a linear ratchet disposed on external portions of members 602, 614, for example, adjacent to gap 620. In such an alternative configuration, the linear ratchet can be used as a gathering member, as described further below.

Although only one projection 616 and one recess 604 is shown in FIGS. 6A-6B, embodiments of the disclosed subject matter are not limited thereto. Rather, the second member 614 can have multiple separate projections 616, and the first member 602 can have multiple corresponding recesses 604. Moreover, although each member 602, 614 is illustrated with only one of the projection 616 or recess 604, members 602, 614 may include both in some embodiments. For example, the second member 614 can have one or more recesses in addition to projection 616, and the first member 602 can have one or more projections in addition to recess 604. In embodiments with multiple projections and rejections, one, some, or all of the projection-recess pairs can include snap-fit features.

Transition of the locking device 600 from the open configuration to the closed configuration can be effected by an actuator. For example, the actuator can be a scissor-style actuator 612, such as that shown in FIG. 6A. The actuator 612 can have a first arm 610 attached to a coupling portion 608 of the first member 602 and a second arm 624 attached to a coupling portion 622 of the second member 614. The first arm 610 and the second arm 624 can be coupled together at hinge 626, which allows arms 610, 624 to rotate with respect to each other. Application of a compressive force at ends 628, 630, which force pushes the ends together, causes the arms 610, 624 to pivot about hinge 626 and thereby urge first member 602 and second member 614 toward each other. The actuator arms 610, 624 can be releasably attached to respective coupling portions 608, 622, such that the actuator 612 can be removed from the locking device 600 after it is placed in the closed configuration, for example, as shown in FIG. 6B. Other styles of actuators are also possible according to one or more contemplated embodiments. Indeed, any actuator capable of operating within the ascending aorta of a patient and of controllably moving the first and second members 602, 614 toward each other while maintaining alignment between recess 604 and projection 616 can be used.

In some embodiments, the actuator 612 defines a region 632 adjacent to gap 620 that is designed to gather part of the leaflet into gap 620 as the locking device 600 is moved from the free end of the leaflet toward the valve annulus. For example, region 632 can be formed by the portion of arms 610, 624 at the hinge 626 that face gap 620. Alternatively or additionally, a separate structure (e.g., a gathering member) can be disposed between the actuator 612 and gap 620 and can be used to gather the leaflet. For example, the gathering member may include a suture, wire, or thread extending between coupling portions 608, 622 in region 632. In another example, gather member may be a bar extending between actuator arms 610, 624 in region 632, with slots that allow the actuator arms 610, 624 to move unrestricted with respect to each other.

As shown in FIG. 6C, the locking device 600, with actuator 612, can be advanced in the open configuration from the ascending aorta 20 to be positioned with respect to one of the leaflet 38 of an existing valvular structure (e.g., a native aortic valve in the illustrated example, or a previously installed prosthetic heart valve in an unillustrated example). For example, the free end 42 of leaflet 38 can be arranged within gap 620 between first member 602 and second member 614. The locking device 600, with actuator 612, can then be further advanced along the leaflet 38, as shown in FIG. 6D. For example, as the locking device 600 is moved toward the valve annulus 18, the region 632 adjacent the hinge 626 of actuator 612 abuts the free end 42 of the leaflet 38 and causes it to fold upon itself, such that folded portion 634 of leaflet 38 is gathered within gap 620. Alternatively or additionally, a gathering member between the gap 620 and region 632 can contact the free end 42 of the leaflet 38 in order to cause the leaflet 38 to fold upon itself as the locking device 600 is advanced.

The amount of locking device advancement can be chosen such that the proximal folded edge 636 of the leaflet 38 is positioned distal to the ostium of coronary artery 22, even when pressed radially outward by subsequent mounting of a prosthetic heart valve within the existing valvular structure. Once the locking device 600 has been sufficiently advanced over the leaflet, the actuator 612 can be actuated to transition the locking device 600 to the closed configuration, as shown in FIG. 6E. The projection of the second member 614 thus moves toward the first member 602, thereby piercing the folded portions of leaflet in gap 620, before being inserted into corresponding recess of the first member 602. Transition to the closed configuration can further lock the first and second members together, for example, via one or more of the snap-fit features described above. The locking of the locking device can ensure that the captured part 634 of the leaflet 38 does not unfold during or after installation of the prosthetic heart valve. The actuator 612 can thus be released from the locking device 600 and can be used to subsequently install a locking device on another leaflet or otherwise retrieved from the patient.

The installation of the locking device 600 thus folds part of leaflet 38 and captures it distal to coronary artery 22, thereby preventing, or at least reducing the risk of obstruction of the ostium of coronary artery 22. If additional capture/folding of one or more leaflets 38 of the existing valvular structure is desired, for example, to prevent, or at least reduce the risk of, obstruction of the ostium of coronary artery 24, then the techniques of FIGS. 6C-6F can be repeated in a similar manner with respect to the next leaflet 38 using another locking device and the same actuator 612 or a different actuator. Although not discussed in detail above, manipulation of the locking device 600 and/or actuator 612 within the patient's anatomy, actuation of actuator 612, and/or decoupling of actuator 612 may be performed using any tools employed in laparoscopic and/or transcatheter heart surgeries.

When no further leaflet capture is desired, a new prosthetic heart valve in a crimped state can subsequently be advanced to the existing valvular structure with leaflets 38 captured by locking devices 600 (such as shown in FIG. 4G). The new valve is disposed within the valvular structure and expanded, such that the captured leaflets 38 are disposed on an external surface of the new valve frame. However, the locking devices 600 retain the captured leaflets 38 in a location distal to the coronary arteries 22, 24, thereby allowing blood to flow from the outflow end of the new prosthetic valve to the coronary arteries 22, 24 once the heart valve implantation is completed.

In some embodiments, a clip member can be used to capture a leaflet in a folded configuration and at a position that avoids obstructing the corresponding coronary ostium. The clip member can have a biased state and a free state. In the biased state, ends of the clip member may be held at opposite ends of the clip member (e.g., having a substantially linear configuration). In the free state, ends of the clip member approach each other such that the clip member at least partially bounds a capture region. The clip member can be advanced in the biased state over a free end of the leaflet. One end of the clip member can be disposed on a distal side of the leaflet. The clip member can then be released from the biased state, thereby transitioning to the free state and capturing a part of the leaflet within the capture region. The new prosthetic heart valve can be subsequently installed.

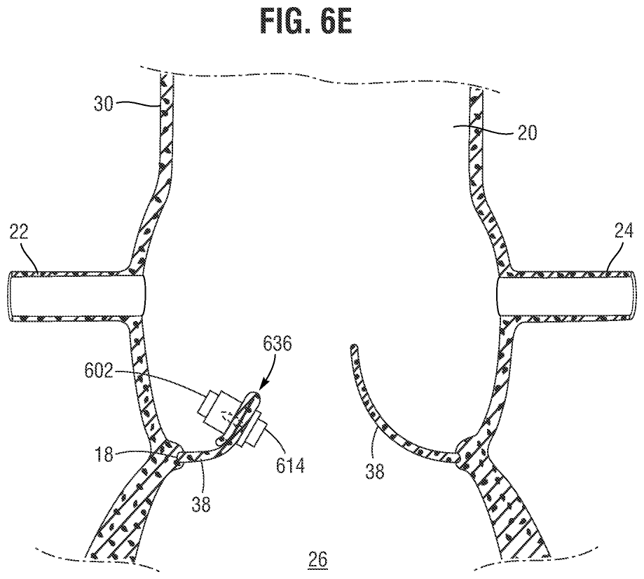

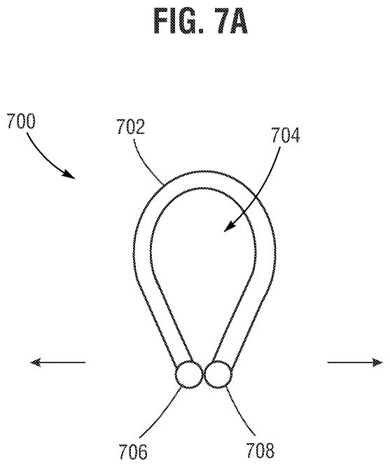

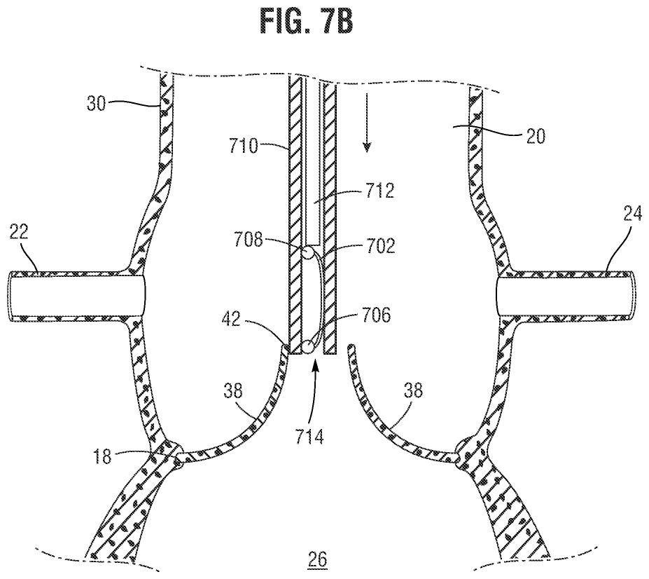

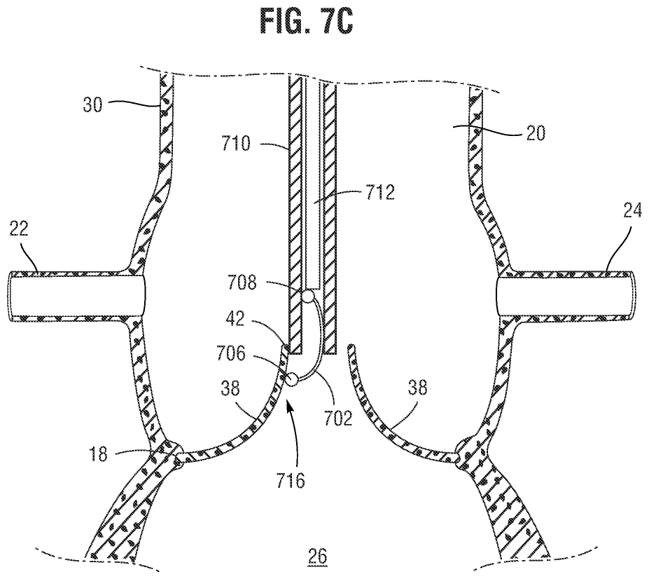

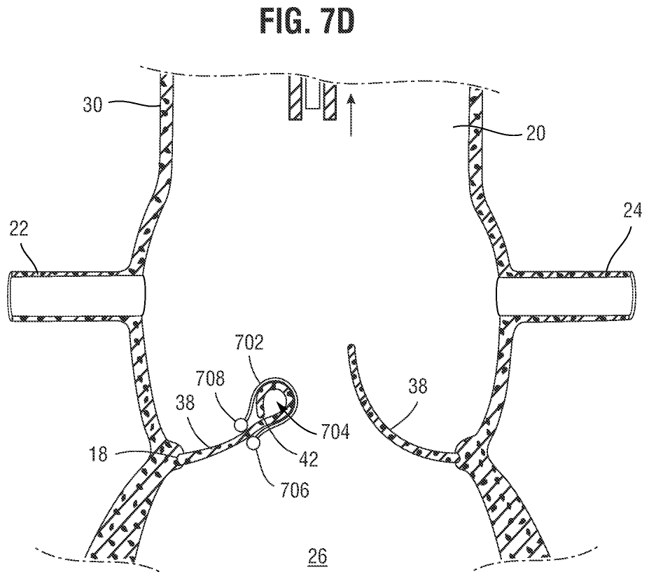

FIG. 7A illustrates an exemplary clip member in a free state, and FIGS. 7B-7D illustrate an exemplary method for folding and/or capturing a leaflet using the exemplary clip member. The clip member 700 can have ends 706, 708 connected together by an intermediate portion 702. In the free state illustrated in FIG. 7A, the clip member 700 has a tear-drop or Ω shape. The intermediate portion 702 adopts a curved configuration that defines a capture region 704 for receiving a leaflet therein. The ends 706, 708 can be in contact with each other or spaced from each other by a gap, which can be sized to be less than a thickness of the leaflet. The clip member 700 can be disposed in the biased state for loading into a delivery catheter and for initial positioning around part of the leaflet. In the biased state illustrated in FIG. 7B, the ends 706, 708 of the clip member 700 are pulled apart such that first end 706 is on a side of intermediate member 702 opposite from that of second end 708. In the biased state, the clip member 700 can adopt a substantially linear or arcuate configuration (e.g., bow-shaped).

The clip member 700 can be formed of a flexible material (e.g., spring metal) or a shape memory alloy that automatically transitions from the biased state to the free state upon release of a force that holds ends 706, 708 apart (e.g., when released from a delivery catheter). For example, the clip member can be formed of steel, cobalt chromium alloy, or nickel titanium alloy (e.g., Nitinol). In some embodiments, the clip member 700 can include a coating on one, some, or all portions thereof. For example, at least a portion of the clip member 700 can have a biocompatible surface coating. Alternatively or additionally, ends 706, 708 can be provided with a surface coating or surface treatment to improve retention to the leaflet once installed. For example, the surface coating or surface treatment may increase a coefficient of friction of ends 706, 708 as compared to a native state of the underlying material.

In some embodiments, a cross-sectional geometry of the clip member from end 706 to end 708 is substantially the same. For example, the clip member 700 can be formed of a substantially constant diameter flexible rod formed into the appropriate curved shape for the free state. In other embodiments, at least ends 706, 708 have a different shape and/or are formed of a different material from that of the intermediate portion 702. For example, the ends 706, 708 can be sized or shaped to assist in retaining the leaflet therebetween. In the illustrated example of FIG. 7A, ends 706, 708 are shaped as balls with diameters greater than a thickness of intermediate portion 702. The ball ends 706, 708 can act to pinch the leaflet therebetween once the clip member 700 transitions to the free state. Alternatively or additionally, the ends 706, 708 can be formed of a magnetic material. When disposed on the leaflet in the free state, ends 706, 708 can attract each other with the leaflet therebetween, which attraction force can improve retention of the clip member 700 to the leaflet. In another example, ends 706, 708 and/or portions of clip member 700 leading up to ends 706, 708 can be formed as extended flat regions that contact the leaflet when disposed thereon in the free state. The extended flat regions can increase the contact surface area, thereby improving the retention of the clip member 700 to the leaflet. Other shapes and configurations for the ends 706, 708 and/or the intermediate portion 702 are also possible according to one or more embodiments of the disclosed subject matter.

As shown in FIG. 7B, the clip member 700 can be disposed within a lumen of delivery catheter 710. The sidewalls of the lumen of the delivery catheter 710 can contact the ends 706, 708 and/or intermediate portion 702 to apply a biasing force that maintains the clip member 700 in the biased state. The delivery catheter 710 can be advanced from the ascending aorta 20 to be positioned with respect to one of the leaflet 38 of an existing valvular structure (e.g., a native aortic valve in the illustrated example, or a previously installed prosthetic heart valve in an unillustrated example). For example, the distal end 714 of the delivery catheter 710 can be disposed within the central gap between leaflets and adjacent to free end 42 of leaflet 38, as illustrated in FIG. 7B. The first end 706 can be extended from the distal end 714 of the delivery catheter 710 and into contact with a distal-side portion 716 of leaflet 38, as illustrated in FIG. 7C. Meanwhile, the sidewalls of the lumen of the delivery catheter 710 can continue to apply a biasing force between end 708 and intermediate portion 702 to maintain the biased state configuration. The positioning of the delivery catheter 710 with respect to the leaflet 38 (as shown in FIG. 7B) can occur at the same time, before, or after the extension of the first end 706 from the end 714 of the delivery catheter.

The clip member 700 can continue to be advanced from the end 714 of the delivery catheter 710 with the first end 706 contacting the distal side of the leaflet 38 until the second end 708 is released from the lumen of the delivery catheter 710. With the lumen no longer applying a biasing force, the clip member 700 is allowed to transition from the biased state to the free state, with ends 706, 708 approaching each other, as shown in FIG. 7D. The second end 708 thus curls distally toward the valve annulus 18 and is disposed on a proximal side of leaflet 38 at a location opposing the first end 706. As the clip member 700 transitions to the free state, the intermediate portion 702 contacts a proximal portion of the leaflet 38 (e.g., the portion adjacent to free end 42), thereby causing the proximal portion to fold upon itself and to be captured within capture region 704. The clip member 700 can be constructed such that the ends 706, 708 in the free state would contact each other or be separated by a gap that is less than a thickness of the leaflet 38. Thus, when the clip member 700 is disposed in the free state on the leaflet 38, a portion of the leaflet 38 between ends 706, 708 is pinched by the ends 706, 708 to retain the clip member 700 in place. The size of the clip member 700 and/or positioning thereof can be chosen such that the leaflet 38 is positioned distal to the ostium of coronary artery 22, even when pressed radially outward by subsequent mounting of a prosthetic heart valve within the existing valvular structure.

In some embodiments, the deployment of the clip member 700 from the delivery catheter 710 can be effected using a deployment member 712. The deployment member 712 may be a flexible rod that can be actuated to move axially within the delivery catheter 710. For example, the deployment member 712 can move distally within the lumen of the delivery catheter 710 to contact the second end 708 and push the clip member 700 from the distal end 714 of the catheter 710. In another example, the clip member 700 can be deployed by maintaining a position of the deployment member 712 with respect to existing valvular structure while retracting the catheter 710 proximally. In still another example, the clip member 700 can be deployed by a combination of movement of the catheter 710 (e.g., retracting proximally) and movement of the deployment member 712 (e.g., moving distally within the lumen of the delivery catheter).

The installation of the clip member 700 thus folds part of leaflet 38 and captures it distal to coronary artery 22, thereby preventing, or at least reducing the risk of obstruction of the ostium of coronary artery 22. If additional capture/folding of one or more leaflets 38 of the existing valvular structure is desired, for example, to prevent, or at least reduce the risk of, obstruction of the ostium of coronary artery 24, then the techniques of FIGS. 7B-7D can be repeated in a similar manner with respect to the next leaflet 38 using another clip member. The same delivery catheter 710 and/or deployment member 712 can be used to deploy the another clip member on the next leaflet 38. For example, two or more clip members 700 in the biased state can be serially disposed within the lumen of the same delivery catheter 710. The deployment member 712, in contact with a second end of a proximal clip member, can push the series of clip members 700 together so as to deploy each clip member sequentially from the end of the catheter 710.

When no further leaflet capture is desired, a new prosthetic heart valve in a crimped state can subsequently be advanced to the existing valvular structure with leaflets 38 captured by clip members 700 (such as shown in FIG. 4G). The new valve is disposed within the valvular structure and expanded, such that the captured leaflets 38 are disposed on an external surface of the new valve frame. However, the clip members 700 retain the capture leaflets 38 in a location distal to the coronary arteries 22, 24, thereby allowing blood to flow from the outflow end of the new prosthetic valve to the coronary arteries 22, 24 once the heart valve implantation is completed.

In some embodiments, the clip member can be plastically deformed to capture a leaflet in in a folded configuration and at a position that avoids obstructing the corresponding coronary ostium. The clip member can have first and second ends connected together by an intermediate portion. In an open configuration of the clip member, the first and second ends can be spaced apart from each other by a gap. In the closed configuration of the clip member, the gap can be reduced or eliminated. The clip member can be positioned in the open configuration such that a free end of the leaflet extends through the gap. The clip member can be move toward a distal portion (e.g., cusp portion) of the leaflet to cause folding of the leaflet. An actuator can then be used to apply a compressive force to the clip member that causes the clip member to plastically deform (e.g., bend) to the closed configuration. At least a folded part of the leaflet can be captured within a region enclosed by the clip member in the closed configuration. The new prosthetic heart valve can be subsequently installed.

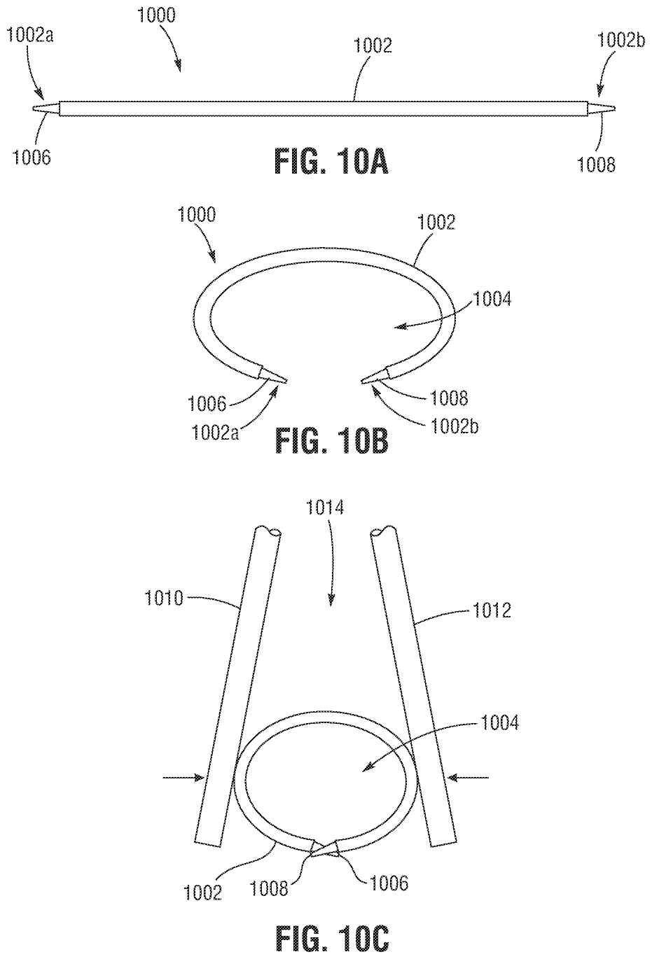

For example, FIG. 10A illustrates a clip member 1000 in an initial configuration, FIG. 10B illustrates the clip member 1000 in an open configuration, and FIG. 10C illustrates the clip member 1000 in a closed configuration. The clip member 1000 can have a first end 1002a and a second end 1002b connected together by an intermediate portion 1002. In the initial configuration illustrated in FIG. 10A, the clip member 1000 has a substantially linear shape, with the first and second ends being on opposite ends of intermediate portion 1002 with respect to a longitudinal axis thereof. Such a configuration may be useful, for example, for delivering the clip member 1000 to an implantation location via a delivery catheter or sheath.

Once delivered to the implantation location, the clip member 1000 may be plastically deformed to the open configuration of FIG. 10B, for example, by bending the intermediate portion 1002 such that ends 1002a, 1002b face each other across a gap 1016 (e.g., to have a substantially C-shape). The clip member 1000 in the open configuration thus defines a partially-enclosed area or capture region 1004. Alternatively, in some embodiments, the clip member 1000 may be initially formed in the open configuration of FIG. 10B, for example, by casting, molding, or otherwise forming the intermediate portion 1002 to have the C-shape without bending from the linear configuration of FIG. 10A.

To transition the clip member 1000 to the closed configuration of FIG. 10C, the clip member 1000 is disposed in an area 1014 between a first arm 1010 and a second arm 1012 of an actuator. The actuator applies a compressive force that plastically deforms the clip member such that the gap 1016 between the first and second ends is eliminated or at least reduced (e.g., less than a thickness of the leaflet), thereby further enclosing capture region 1004 to capture a part of the leaflet therein. The actuator can be, for example, tweezers, surgical tongs, or a scissor-style actuator (e.g., similar to actuator 612 illustrated in FIG. 6A). However, other actuators are also possible according to one or more contemplated embodiments. Indeed, the actuator can be any type of actuator capable of operating within the ascending aorta of a patient and of applying a compressive force to the clip member. For example, the arms 1010, 1012 can be coupled to opposite ends of the clip member 1000 and a sheath can be advanced over the arms from a proximal end thereof toward a distal end thereof, thereby urging the arms together to apply the compressive force to the clip member.

The clip member 1000 can be formed of a material having sufficient rigidity to maintain its shape after being plastically deformed. For example, the clip member 1000 can comprise a metal, metal-alloy, or any combination thereof, such as steel or cobalt chromium alloy. In some embodiments, the clip member 1000 can include a coating on one, some, or all portions thereof. For example, at least a portion of the clip member 1000 can have a biocompatible surface coating. Alternatively or additionally, ends 1002a, 1002b (e.g., tip 1006 and/or tip 1008) can be provided with a surface coating or surface treatment to improve retention to the leaflet once installed. For example, the surface coating or surface treatment may increase a coefficient of friction of tips 1006, 1008 as compared to a native state of the underlying material.

In some embodiments, a cross-sectional geometry of at least the intermediate portion between ends 1002a, 1002b can be substantially the same. For example, the clip member 1000 can be formed of a substantially constant diameter rod plastically deformed from the linear configuration of FIG. 10A to the open configuration of FIG. 10B. In some embodiments, at least ends 1002a, 1002b can have a different shape and/or are formed of a different material from that of the intermediate portion 1002. For example, the ends 1002a, 1002b can be sized or shaped to assist in retaining the leaflet therebetween. In the illustrated example of FIGS. 10A-10C, the first end 1002a has a first tip 1006 and the second end 1002b has a second tip 1008. Each tip 1006, 1008 can taper in a direction away from the intermediate portion 1002 to a sharp point or at least a narrowed-diameter end, so as to be able to pierce through the leaflet when contacting therewith. Alternatively, in some embodiments, tips 1006, 1008 can be replaced with ball ends that act to pinch the leaflet therebetween when the clip member 1000 is in the closed configuration, similar to the ball ends illustrated in FIG. 7A. Alternatively or additionally, the tips 1006, 1008 and/or ends 1002a, 1002b can be formed of a magnetic material. When disposed on the leaflet in the open configuration, the magnetic material can attract each other with the leaflet therebetween, which attraction force can improve retention of the clip member 1000 to the leaflet. Further shapes and configurations for the ends 1002a, 1002b and/or the intermediate portion 1002 are also possible according to one or more embodiments of the disclosed subject matter.

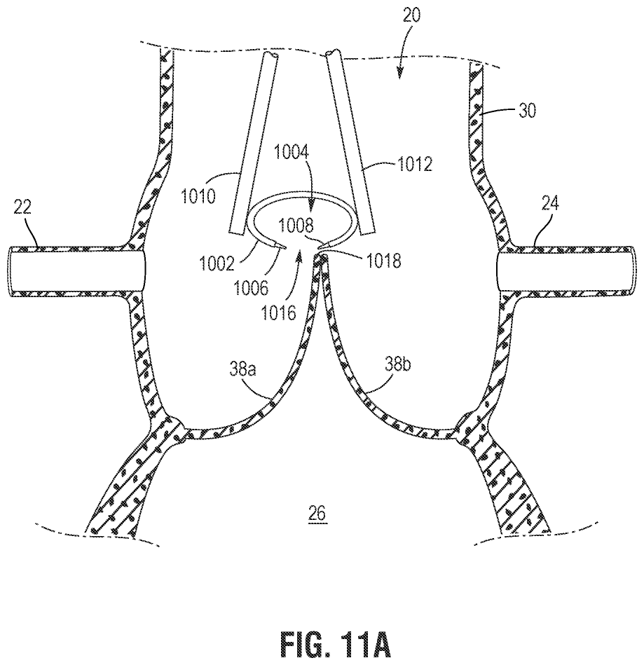

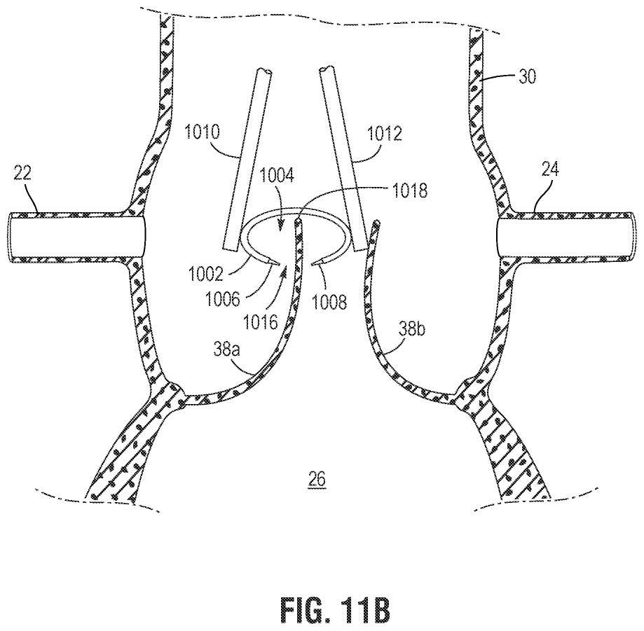

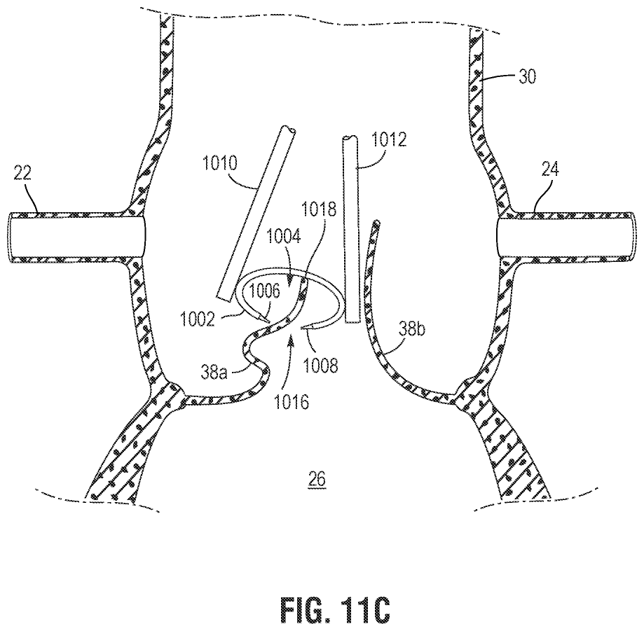

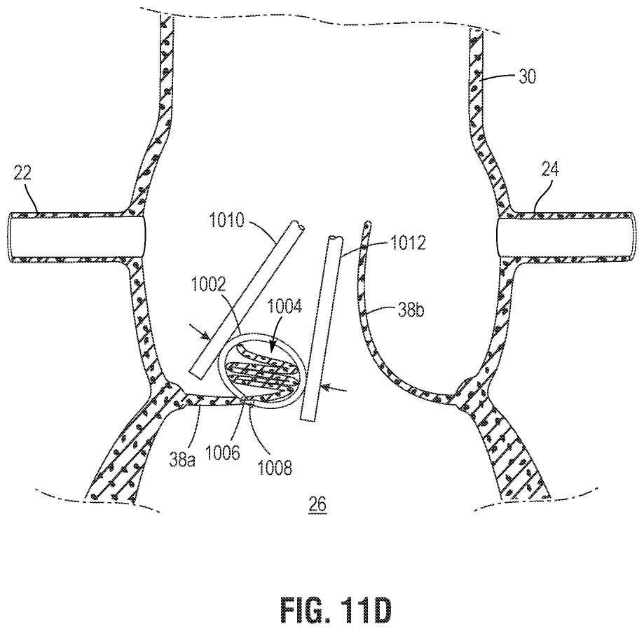

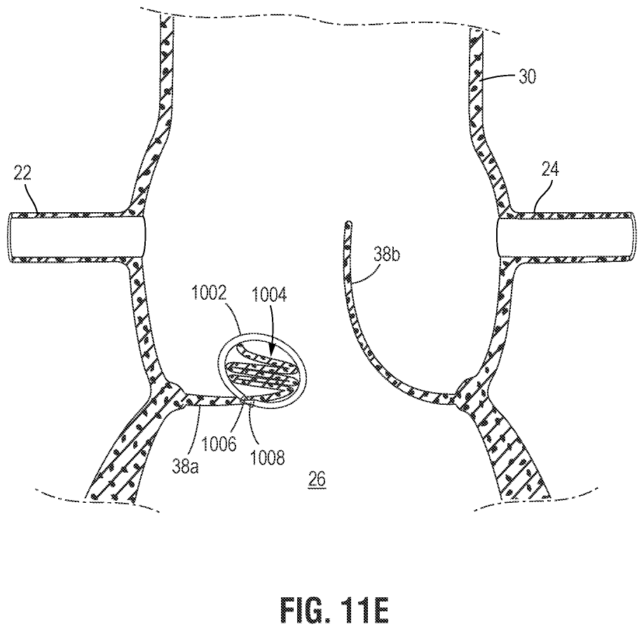

As shown in FIG. 11A, actuator arms 1010, 1012 can be used to advance the clip member in the open configuration from the ascending aorta 20 so as to be positioned with respect to one of the leaflet 38a of an existing valvular structure (e.g., a native aortic valve in the illustrated example, or a previously installed prosthetic heart valve in an unillustrated example). For example, the free end 1018 of leaflet 38a can be arranged to extend through gap 1016 between the first tip 1006 and the second tip 1008 into the capture region 1004, as shown in FIG. 11B. The actuator arms 1010, 1012 can then be used to further advance the clip member along the leaflet 38a, as shown in FIG. 11C. For example, as the clip member is moved toward a distal portion of the leaflet 38a (e.g., a root or cusp region of the leaflet, or an annulus of the valvular structure), the intermediate portion 1002 of the clip member abuts the free end 1018 of the leaflet 38a and causes it to fold upon itself, such that folded portion of leaflet 38a is gathered within gap 1016.

The amount of clip member advancement can be chosen such that the folded leaflet 38a is positioned distal to the ostium of coronary artery 22, even when pressed radially outward by subsequent mounting of a prosthetic heart valve within the existing valvular structure. Once the clip member has been sufficiently advanced over the leaflet, the actuator can be actuated to apply a compressive force between arms 1010, 1012, thereby plastically deforming the clip member to the closed configuration, as shown in FIG. 11D. The tips 1006, 1008 of the clip member thus move toward each other and pierce, or at least clamp, a portion of the leaflet 38a therebetween. The plastic deformation of the clip member can be effective to lock the tips 1006, 1008 in position within respect to the leaflet 38a. The actuator can then be released from the clip member and can be used to subsequently install a clip member on another leaflet or otherwise retrieved from the patient. After removal of the actuator, the clip member remains behind in place on the folded leaflet 38a, as shown in FIG. 11E. The plastic deformation of the clip member can thus ensure that the part of the leaflet 38a captured within region 1004 does not unfold during or after installation of the prosthetic heart valve.

It should be understood that only a portion of the actuator arms 1010, 1012 are shown in FIGS. 11A-11D for clarity of illustration. However, in practical embodiments, the actuator would include additional portions not illustrated, such as a hinge section (e.g., for scissor-style actuators), a joint section (e.g., for tweezer or surgical tongs), and/or a delivery catheter or sheath (e.g., for a sheath advanced over the arms to provide a force urging the arms together). Moreover, one or more delivery catheters or sheaths can be provided to deliver the clip member and the actuator to the ascending aorta, and/or for operation of the actuator within the ascending aorta.

The installation of the clip member thus folds part of leaflet 38a and captures it distal to coronary artery 22, thereby preventing, or at least reducing the risk of obstruction of the ostium of coronary artery 22. If additional capture/folding of one or more leaflets 38 of the existing valvular structure is desired, for example, to prevent, or at least reduce the risk of, obstruction of the ostium of coronary artery 24, then the techniques of FIGS. 11A-11E can be repeated in a similar manner with respect to the next leaflet 38 using another clip member and the same actuator (e.g., arms 1010, 1012) or a different actuator. Although not discussed in detail above, manipulation of the clip member 1000 and/or the actuator within the patient's anatomy, actuation of the actuator to plastically deform clip member 1000, and/or decoupling of the actuator from the clip member 1000 may be performed using any tools employed in laparoscopic and/or transcatheter heart surgeries.

When no further leaflet capture is desired, a new prosthetic heart valve in a crimped state can subsequently be advanced to the existing valvular structure with leaflets 38 captured by clip member 1000. The new valve is disposed within the valvular structure and expanded, such that the captured leaflets 38 are disposed on an external surface of the new valve frame. However, the clip members retain the captured leaflets 38 in locations distal to the coronary arteries 22, 24, thereby allowing blood to flow from the outflow end of the new prosthetic valve to the coronary arteries 22, 24 once the heart valve implantation is completed.

In some embodiments, a tool can be used to temporarily fold a leaflet below the ostia of the coronary artery and hold it in place until a prosthetic heart valve can be positioned within the existing valvular structure and at least partially expanded therein. As the prosthetic heart valve expands, the folded leaflet is pushed by the valve frame wall toward and into contact with the surrounding anatomy (e.g., the aortic wall) or pre-existing structure (e.g., the valve frame wall of a previously implanted prosthetic valve). Prior to full expansion of the prosthetic heart valve (e.g., before a size of an annular region between the valve frame and the surrounding structure would prevent removal of the tool), the portion of the tool coupled to the leaflet can be pulled away therefrom. Since the leaflets have been positioned distally and folded, the risk of curtaining of the coronary ostia during valve expansion can be reduced.

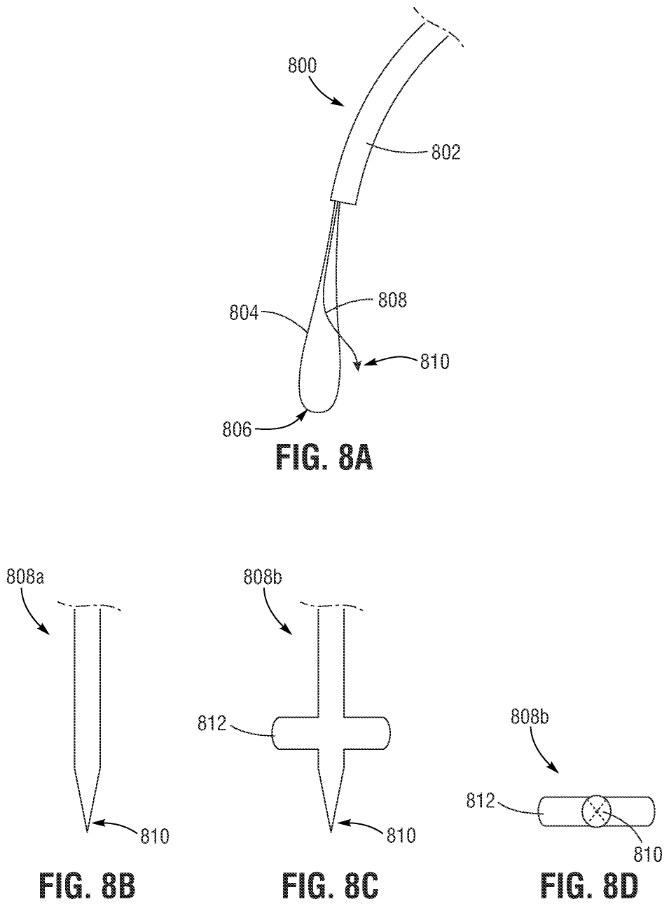

For example, FIG. 8A illustrates an exemplary leaflet folding tool 800 that can be used to temporarily fold a leaflet of an existing valvular structure. The tool 800 comprises a delivery catheter or sheath 802 with a distal end configured to be disposed within an ascending aorta of a patient. The sheath 802 can contain at least two members—a positioning member 804 and a leaflet engagement member 808. Each of the members 804, 808 can be independently maneuvered within sheath 802 and extending from sheath 802, for example, via operation of a handle (not shown) at the proximal region of the sheath 802 by an operator. The sheath 802 in the illustrated embodiment is in the form of a shaft, although in other embodiments, the sheath can include multiple shafts, which can be disposed adjacent to each other within the same sheath.

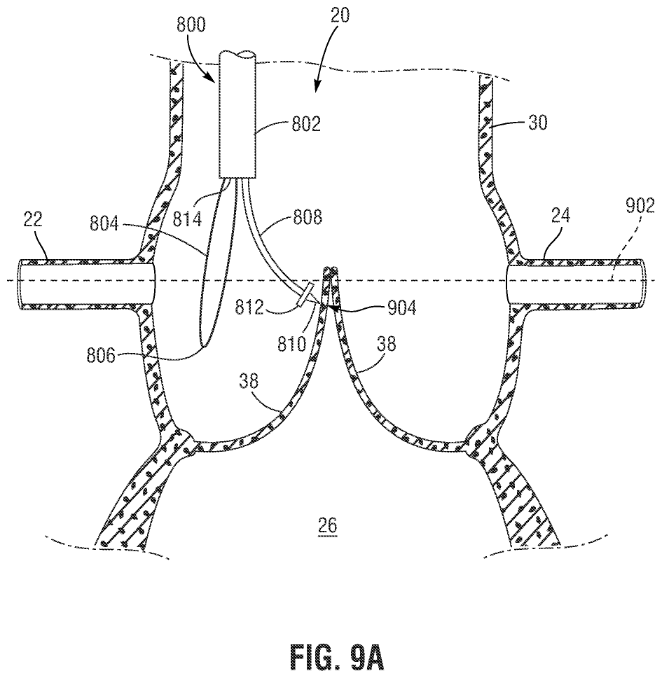

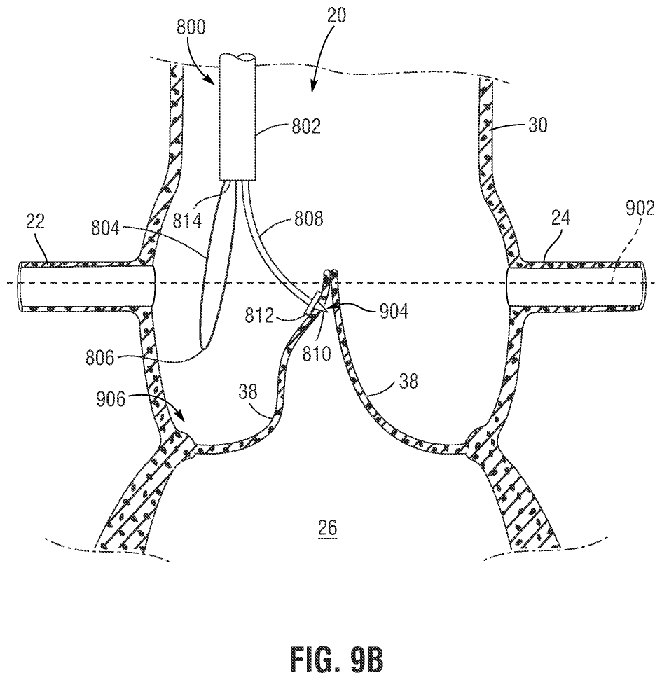

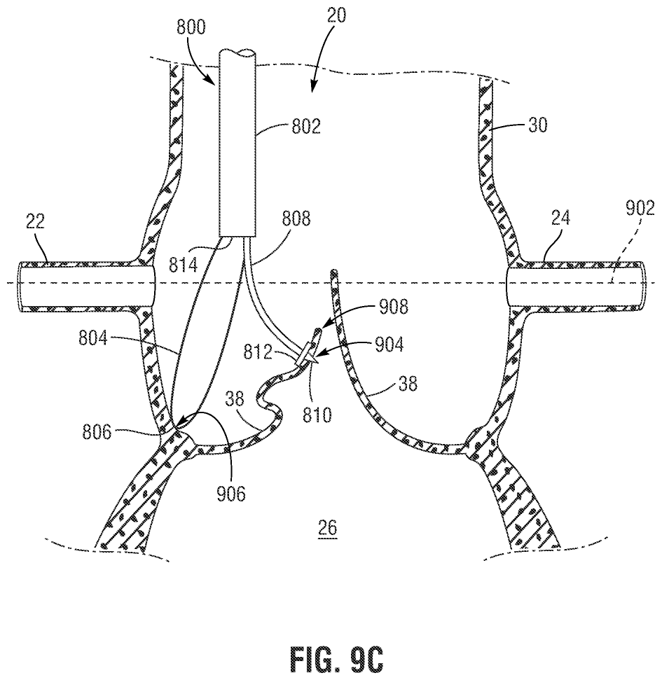

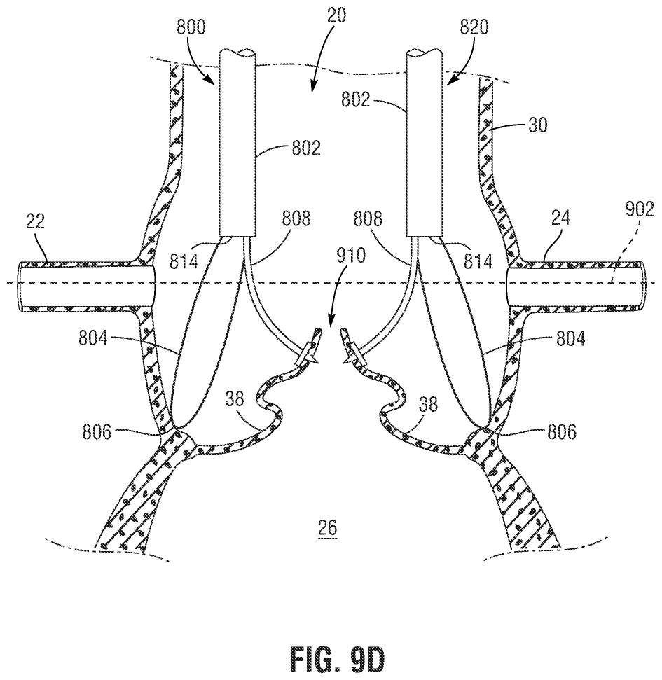

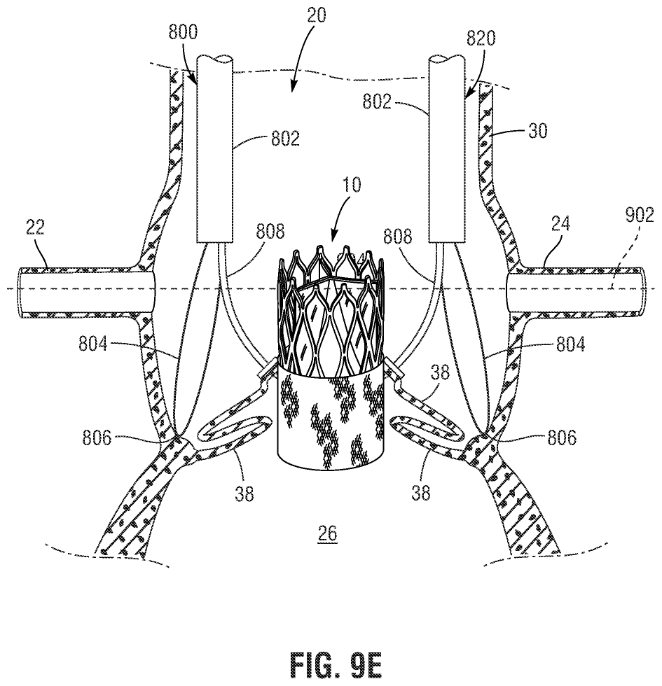

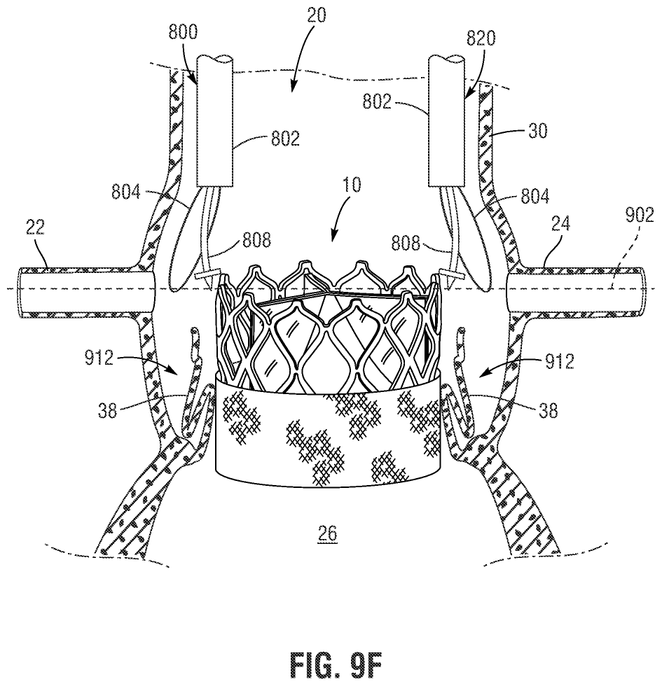

Each of the members 804, 808 can be extended from the distal end of the sheath 802 to interact with the existing valvular structure. For example, as shown in FIGS. 9A-9F, the positioning member 804 can be configured for insertion into a pocket or sinus 906 between a leaflet 38 of the existing valvular structure and the frame 12 (or the aortic wall 30 when dealing with the native valve), in order to guide placement and positioning of the leaflet engagement member 808 with respect to leaflet 38. Each of the members 804, 808 can be a pre-shaped wire or cable, for example, a wire formed of a shape memory material, such as Nitinol. Thus, as the members 804, 808 are advanced out of the distal end of the sheath, end portions thereof can adopt their pre-determined shape. Alternatively, positioning member 804 and/or leaflet engagement member 808 can be formed of other materials, such as a metal (e.g., steel, titanium, etc.), metal alloy (e.g., cobalt chromium alloy, etc.), plastic, or any combination thereof.

In some embodiments, at least a distal-most end portion 806 of the positioning member 804 is constructed to be atraumatic (e.g., blunt or otherwise lacking sharp edges) to avoid damage to the surrounding anatomy during operation. For example, the positioning member 804 can have a contoured end portion 806 having a circular shape, oval shape (e.g., spoon-shaped), elliptical shape, C-shape, J-shape, or any other arcuate shape, such that an area of contact between the end portion 806 and a base (e.g., cusp portion) of the leaflet can be increased. In some embodiments, the positioning member 804 can be formed from a plurality of wires, for example, as a pair of wires that are bent laterally to converge to contact or attach to each other at end portion 806. Alternatively, in some embodiments, the positioning member comprises a catheter or sheath with an atraumatic distal end.

The leaflet engagement member 808 can comprise a spiked tip 810 at an axial end thereof, which is constructed to penetrate or otherwise grasp a leaflet. In some embodiments, the spiked tip 810 simply includes a sharp point or spike, for example, as with member 808a illustrated in the detail view of FIG. 8B. The sharp point can be used to pierce a portion of the leaflet in order to engage therewith. In other embodiments, the tip can have a barbed configuration that prevents or at least resists further insertion of the tip through the leaflet, so as to assist in pushing the leaflet distally. For example, in the detail views of FIGS. 8C-8D, leaflet engagement member 808b has a barbed configuration with laterally-extending base 812 being spaced proximally from the spiked tip 810 along an axial direction of the member 808b. Alternatively, the barbed configuration can comprise an annular base (e.g., a radially-extending base) instead of the laterally-extending base 812.

FIGS. 9A-9C show various stages for using the leaflet folding tool 800 to distally position a leaflet 38 of the existing valvular structure to avoid coronary artery obstruction by subsequent implantation of a prosthetic heart valve. Delivery shaft 802 can be advanced to the existing valvular structure from the ascending aorta 20. The positioning member 804 and the leaflet engagement member 808 may be retained within the shaft 802 prior to reaching the existing valvular structure. Once the shaft 802 reaches the existing valvular structure (e.g., the distal end 814 is positioned near the coronary artery 22), the positioning member 804 can be advanced from the distal end 814 of the shaft 802 toward, but spaced from, a base 906 of the leaflet 38 (e.g., the cusp floor of the leaflet). Simultaneous with the advancement of the positioning member 804 or sequential thereto (e.g., before or after), the leaflet engagement member 808 can be advanced from the distal end 814 of the shaft toward a proximal portion 904 of the leaflet 38, as shown in FIG. 9A.

The leaflet engagement member 808 can be pushed into contact with the leaflet portion 904, such that the spiked tip 810 penetrates therethrough (e.g., from a proximal side thereof to a distal side thereof), as shown in FIG. 9B. The lateral base 812 is brought into contact with the proximal side of the leaflet portion 904 to restrict further insertion of the spiked tip 810. The lateral base 812 also increases the surface contact area to assist in pushing the leaflet distally. In particular, the leaflet engagement member 808 and the positioning member 804 can be advanced together (e.g., by moving delivery shaft 802 distally, by simultaneously advancing members 804, 808 distally out of shaft 802, or by any combination thereof), such that the engagement member 808 pushes the free end 908 of the leaflet away from the plane 902 of the coronary ostia, effectively folding the leaflet, as shown in FIG. 9C. The advancement of both members 804, 808 can continue until the positioning member 804 reaches the base portion 906 of the leaflet 38. With the leaflet 38 held in this folded position, a prosthetic heart valve can be subsequently implanted, whereby the folded leaflet 38 reduces the risk of obstruction the ostium of coronary artery 22 after valve implantation.

In some embodiments, at least two of the leaflets of the existing valvular structure can be subjected to folding, for example, using a respective leaflet folding tool. For example, a leaflet folding tool can be provided for each leaflet (e.g., three folding tools for three leaflets). If folding of additional leaflets of the existing valvular structure is desired, for example, to prevent, or at least reduce the risk of, obstruction of the ostium of coronary artery 24, then the techniques of FIGS. 9A-9C can be repeated in a similar manner with respect to the next leaflet 38 using a separate folding tool 820, for example, as shown in FIG. 9D. In such repetition, the portion 904 of each leaflet 38 folded by the respective tool 800, 820 can be circumferentially aligned with the ostium of coronary artery 22 or coronary artery 24 (e.g., aligned or substantially aligned along a same radial vector extending from a center of the existing valvular structure, as viewed from the ascending aorta 20).

When no further leaflet folding is desired, a new prosthetic heart valve in a crimped state can subsequently be advanced to the existing valvular structure with captured leaflets 38. For example, as depicted in FIG. 9E, a new prosthetic valve 10 (which can be the valve 10 of FIG. 2A or a different prosthetic valve) can be positioned in region 910 between free ends of the leaflets 38, as shown in FIG. 9E. The new prosthetic valve 10 is then partially expanded such that the folded leaflets 38 are disposed in an annular region 912 between the external circumferential surface of the new valve frame and the surrounding structures (e.g., the aortic wall), as shown in FIG. 9F. Prior to full expansion of the valve 10, the leaflet engagement members 808 can be removed from the respective leaflets 38 (e.g., by retracting the leaflet engagement members proximally) and the members 804, 808 removed from annular region 912, thereby allowing the valve 10 to fully expand toward the aortic wall 30. Once disengaged from the leaflets 38, the leaflet folding tools 800, 820 can be removed from the ascending aorta, for example, by retracting members 804, 808 into the respective sheath 802 and removing from the vasculature of the patient. The folded leaflets are thus pressed sideways against the aortic wall 30 by the fully-expanded frame of prosthetic heart valve 10 at a location distal to the plane 902 of the coronary ostia, thereby reducing the risk of obstruction of the coronary arteries 22, 24 and allowing blood to flow from the outflow end of the new prosthetic valve to the coronary arteries 22, 24 once the heart valve implantation is completed.

In any of the above noted examples, any of the delivery catheters or sheaths, locking devices, folding tools, and/or clip members can be provided from the ascending aorta to the existing valvular structure via any transcatheter aortic access route, such as but not limited to transfemoral, transaxillary, transaortic, transapical, transcarotid, transseptal, transcaval, subclavian, radial, or carotid approaches.

In any of the above noted examples, any of the delivery catheters or sheaths, locking devices, folding tools, and/or clip members can be used to capture/fold leaflets of the native valvular structure of another of the native heart valves, for example, pulmonary, tricuspid, or mitral valves, or prosthetic heart valves previously implanted therein. It should be noted that any of the devices or techniques described herein in connection with folding or otherwise modifying the position of a native leaflet to avoid or minimize blockage of the coronary ostia can be applied to one or more leaflets of a previously implanted prosthetic heart valve prior to or while implanting a new prosthetic heart valve in the previously implanted prosthetic heart valve in a valve-in-valve procedure. Further, any of the delivery catheters or sheaths, locking devices, folding tools, and/or clip members can thus be configured to be delivered via other blood vessels to the heart. For example, to access the tricuspid or pulmonary positions, the delivery catheters or sheaths, locking devices, folding tools, and/or clip members can be delivered via the inferior and superior vena cava. For accessing the mitral position, the delivery catheters or sheaths, locking devices, folding tools, and/or clip members can be delivered via a transseptal procedure, for example, by advancing through the inferior or superior vena cava and across through the atrial septum. Alternatively, for accessing the mitral position, the delivery catheters or sheaths, locking devices, folding tools, and/or clip members can be delivered via a transapical approach, for example, by advancing through the wall of the left ventricle at the base of the heart.

In any of the above noted examples, the capture/folding of leaflets in the existing valvular structure can be performed as part of, before, or after a balloon annular valvuloplasty procedure on the existing valvular structure, for example, to prepare an existing heart valve (e.g., native aortic valve or previously implanted prosthetic valve) for subsequent implantation of a new prosthetic valve. Alternatively, in any of the above noted examples, the capture/folding of leaflets in the existing valvular structure can be performed as part of or preceding a transcatheter aortic valve implantation (TAVI) or transcatheter aortic valve replacement (TAVR) procedure, for example, to replace a native aortic valve or to replace a failing prosthetic valve (e.g., ViV procedure). Indeed, while the examples of FIGS. 3A-11E specifically illustrate capture/folding of leaflets in a native aortic valve, it should be readily understood that the same techniques can be applied to previously-implanted prosthetic heart valves, for example, prior to or simultaneous with the mounting of a new prosthetic heart valve within the previously-implanted heart valve. Accordingly, embodiments of the disclosed subject matter are not limited to the particular illustrations.

Where not otherwise expressly noted above, components can be formed of any type of biocompatible material of sufficient strength or flexibility for use in the particular application. Such materials can include, but are not limited to, metals or metal alloys such as surgical steel, titanium, cobalt chromium alloy, and nickel titanium alloy (nitinol), and polymers such as polyurethane, polytetrafluorethylene, and polyethersulfone.

### Additional Examples of the Disclosed Technology

In view of the above described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A method comprising:

providing a crimped prosthetic heart valve in an ascending aorta of a patient, the prosthetic heart valve having an expandable frame supporting a first valvular structure therein and a leaflet capture member disposed along an exterior of the expandable frame and coupled at opposite ends thereof to the expandable frame, the leaflet capture member extending along an axial direction of the frame and comprising a distal portion, a proximal portion, and an intermediate portion between and coupled to the distal and proximal portions by respective connecting portions,

positioning the crimped prosthetic heart valve within an existing second valvular structure such that a free end of a leaflet of the second valvular structure contacts the leaflet capture member on an exterior of the expandable frame, the second valvular structure being between the ascending aorta and a left ventricle of a heart; and

expanding the expandable frame of the prosthetic heart valve so as to mount the prosthetic heart valve within the second valvular structure, the expanding being such that a shape of the leaflet capture member deforms to capture the free end of the leaflet between the intermediate portion and the distal portion,

wherein the captured free end of the leaflet allows blood to flow through a frame of the prosthetic heart valve to a coronary artery of the heart, which would otherwise be blocked by the leaflet of the second valvular structure.

Example 2. The method of any example herein, particularly Example 1, wherein a plurality of the leaflet capture members is at different locations along a circumference of the expandable frame, and the expanding is such that a free end of each leaflet of the existing second valvular structure is simultaneously captured by a respective one of the leaflet capture members.

Example 3. The method of any example herein, particularly any one of Examples 1-2, wherein, prior to expanding the expandable frame, each leaflet capture member comprises a substantially straight bar extending along the axial direction.

Example 4. The method of any example herein, particularly any one of Examples 1-3, wherein at least the proximal portion of each leaflet capture member has an open cell structure that allows blood to flow therethrough.

Example 5. The method of any example herein, particularly any one of Examples 1-4, wherein at least one of the connecting portions comprises a region with a narrowed cross-section compared to the distal portion, the proximal portion, and/or the intermediate portion.

Example 6. The method of any example herein, particularly any one of Examples 1-5, wherein at least one of the connecting portions comprises a weakened, notched, or grooved portion or a region with a narrowed cross-section.

Example 7. The method of any example herein, particularly any one of Examples 1-6, wherein:

before the expanding, a first connecting portion that couples the proximal portion to the intermediate portion is disposed proximal to a second connecting portion that couples the distal portion to the intermediate portion; and

after the expanding, the first connecting portion is disposed distal to the second connecting portion.

Example 8. The method of any example herein, particularly any one of Examples 1-7, wherein the existing second valvular structure is a native aortic valve of the heart.

Example 9. The method of any example herein, particularly any one of Examples 1-7, wherein the existing second valvular structure is of a second prosthetic valve previously implanted in the patient, and the expanding is such that the prosthetic heart valve is installed within the second prosthetic valve.

Example 10. A prosthetic heart valve comprising:

an annular frame that is expandable from a crimped state to a deployed state;

a first valvular structure formed by a leaflet assembly, the leaflet assembly comprising a plurality of leaflets, the first valvular structure being disposed within and attached to the annular frame; and

a leaflet capture member disposed along an exterior of the annular frame and coupled at opposite ends thereof to the annular frame, the leaflet capture member extending along an axial direction of the annular frame, the leaflet capture member comprising a distal portion, a proximal portion, and an intermediate portion between and coupled to the distal and proximal portions by respective connecting portions,

wherein the leaflet capture member is configured to change shape upon expansion of the annular frame to the deployed state so as to capture a free end of a leaflet of an existing second valvular structure between the intermediate portion and the distal portion.

Example 11. The prosthetic heart valve of any example herein, particularly Example 10, further comprising additional leaflet capture members disposed at different locations along a circumference of the annular frame.

Example 12. The prosthetic heart valve of any example herein, particularly any one of Examples 10-11, wherein each leaflet capture member comprises a substantially straight bar extending along the axial direction prior to expansion of the annular frame.

Example 13. The prosthetic heart valve of any example herein, particularly any one of Examples 10-12, wherein at least the proximal portion of each leaflet capture member has an open cell structure that allows blood to flow therethrough.

Example 14. The prosthetic heart valve of any example herein, particularly any one of Examples 10-13, wherein at least one of the connecting portions comprises a weakened region.

Example 15. The prosthetic heart valve of any example herein, particularly any one of Examples 10-14, wherein at least one of the connecting portions comprises a region with a narrowed cross-section compared to the distal portion, the proximal portion, and/or the intermediate portion.

Example 16. The prosthetic heart valve of any example herein, particularly any one of Examples 10-15, wherein at least one of the connecting portions comprises a notched or grooved region.

Example 17. The prosthetic heart valve of any example herein, particularly any one of Examples 10-16, wherein the leaflet capture member is formed of metal or a biocompatible polymer.

Example 18. The prosthetic heart valve of any example herein, particularly any one of Examples 10-17, wherein the leaflet capture member is configured such that:

prior to expansion of the annular frame, a first connecting portion that couples the proximal portion to the intermediate portion is disposed proximal to a second connecting portion that couples the distal portion to the intermediate portion; and

after expansion of the annular frame, the first connecting portion is disposed distal to the second connecting portion.

Example 19. A method comprising:

(a) passing a first portion of a suture through a first portion of a leaflet of an existing valvular structure so as to extend from a proximal side of the leaflet, which faces an ascending aorta of a patient, to a distal side of the leaflet, which faces a left ventricle of a heart of the patient;

(b) after (a), conveying the first portion of the suture back to the ascending aorta via a central gap between free ends of the leaflets of the valvular structure;

(c) after (b), forming a knot by tying the first portion of the suture to a second portion of the suture disposed proximal to the first portion of the leaflet, such that part of the leaflet between the first portion of the leaflet and the leaflet free end is disposed within a loop formed between the tied first and second suture portions; and

(d) sliding at least one of the knot and the suture with respect to the other, so as to reduce a size of the loop, thereby compacting the leaflet part by moving the leaflet free end toward the first portion of the leaflet.

Example 20. The method of any example herein, particularly Example 19, wherein the compacting in (d) is such that the leaflet part within the loop folds on itself.

Example 21. The method of any example herein, particularly any one of Examples 19-20, further comprising repeating (a)-(d) with respect to another leaflet of the valvular structure.

Example 22. The method of any example herein, particularly any one of Examples 19-21, further comprising:

after (d), mounting a prosthetic heart valve within the existing valvular structure,

wherein the compacted leaflet part allows blood to flow through a frame of the prosthetic heart valve to a coronary artery of the heart, which would otherwise be blocked by the leaflet of the existing valvular structure.

Example 23. The method of any example herein, particularly any one of Examples 19-22, wherein (a) includes piercing the first leaflet portion with a needle coupled to the first suture portion.

Example 24. The method of any example herein, particularly Example 23, further comprising:

prior to (a), providing a delivery catheter in the ascending aorta, and

positioning the delivery catheter with an end thereof adjacent to the first leaflet portion,

wherein (a) comprises extending the needle, with first suture portion attached, from the delivery catheter end to pierce the first leaflet portion.

Example 25. The method of any example herein, particularly Example 24, further comprising:

after (a) and prior to (c), withdrawing the delivery catheter into the ascending aorta to expose the second suture portion.

Example 26. The method of any example herein, particularly any one of Examples 19-25, wherein (d) comprises moving a sliding member distally along said suture to push the knot toward the first leaflet portion.

Example 27. The method of any example herein, particularly Example 26, wherein the sliding member comprises a portion of the delivery catheter.

Example 28. The method of any example herein, particularly Example 26, wherein the sliding member is extended from an end of the delivery catheter.

Example 29. The method of any example herein, particularly any one of Examples 19-25, wherein (d) comprises using a positioning member to abut a proximal end of the knot while proximally retracting the suture.

Example 30. The method of any example herein, particularly any one of Examples 26-29, further comprising, after (d), using the sliding member or the positioning member to lock a position of the knot relative to the first leaflet portion.

Example 31. The method of any example herein, particularly any one of Examples 19-30, further comprising, after (d), locking a position of the knot relative to the first leaflet portion.

Example 32. The method of any example herein, particularly Example 31, wherein the locking comprises:

disposing a separate locking member into contact with the knot;

tightening the knot such that the knot is incapable of sliding along the suture;

bonding, fusing, or encasing the knot; or

any combination of the above.

Example 33. The method of any example herein, particularly any one of Examples 19-32, wherein the first leaflet portion is distal to an ostium of a coronary artery of the heart.

Example 34. The method of any example herein, particularly any one of Examples 19-33, wherein the first leaflet portion is closer to a distal attachment portion of the leaflet than a center of the existing valvular structure.

Example 35. The method of any example herein, particularly any one of Examples 19-34, wherein the existing valvular structure is a native aortic valve of the heart.

Example 36. The method of any example herein, particularly any one of Examples 19-34, wherein the existing valvular structure is of a second prosthetic heart valve previously implanted in the patient, and the mounting the prosthetic heart valve comprises installing the prosthetic heart valve within the second prosthetic heart valve.

Example 37. A method comprising:

(a) at a first portion of a leaflet of an existing valvular structure, forming one or more first anchors for a first suture, the first suture extending proximally from the one or more first anchors at the first leaflet portion, the existing valvular structure being between an ascending aorta and a left ventricle of a heart;

(b) at a second portion of the leaflet, forming one or more second anchors for a second suture, the second suture extending proximally from the one or more second anchors at the second leaflet portion, the second leaflet portion being closer to a free end of the leaflet than the first leaflet portion, one of the first and second leaflet portions is closer to a free end of the leaflet than the other of first and second leaflet portions; and

(c) pulling said one of the first and second leaflet portions toward the other of the first and second leaflet portions so as to compact part of the leaflet disposed between the first and second anchors.

Example 38. The method of any example herein, particularly Example 37, wherein:

the one or more first anchors is a pair of first anchors, one of the first anchors being formed on a distal side of the leaflet, the other of the first anchors being formed on a proximal side of the leaflet,

the one or more second anchors are a pair of second anchors, one of the second anchors being formed on the distal side of the leaflet, the other of the second anchors being formed on the proximal side of the leaflet,

the distal side faces the left ventricle, and

the proximal side faces the ascending aorta.

Example 39. The method of any example herein, particularly any one of Examples 37-38, wherein (c) comprises distally sliding a coupling member along the first and second sutures from the ascending aorta toward the leaflet.

Example 40. The method of any example herein, particularly Example 39, wherein:

the coupling member has a first conduit through which the first suture extends and a second conduit through which the second suture extends, and

a spacing between the first and second conduits is less than a distance along the leaflet between the first leaflet portion and the second leaflet portion.

Example 41. The method of any example herein, particularly Example 39, wherein:

the coupling member has a single conduit through which the first and second suture extend, and

a size of the conduit is less than a distance along the leaflet between the first and second leaflet portions.

Example 42. The method of any example herein, particularly any one of Examples 39-41, further comprising, after (c), locking a position of the coupling member relative to the first and second anchors.

Example 43. The method of any example herein, particularly any one of Examples 37-42, wherein one or more of the formed first and second anchors comprises a suture clip or fastener.

Example 44. The method of any example herein, particularly any one of Examples 37-43, wherein one or more of the formed first and second anchors comprises one or more plications of the leaflet.

Example 45. The method of any example herein, particularly Example 44, wherein each plication includes a locking member that maintains a position of the plication with respect to the corresponding first or second leaflet portion.

Example 46. The method of any example herein, particularly any one of Examples 37-45, wherein (a) comprises:

positioning a first delivery catheter with an end thereof adjacent to a proximal side of the first leaflet portion;

piercing the first leaflet portion with a needle coupled to the first suture by extending the needle from the end of the first delivery catheter;

forming one of the first anchors on a distal side of the first leaflet portion; and

forming another of the first anchors on the proximal side of the first leaflet portion.

Example 47. The method of any example herein, particularly Example 46, wherein (a) further comprises, after the piercing, proximally retracting the first delivery catheter toward the ascending aorta.

Example 48. The method of any example herein, particularly any one of Examples 46-47, wherein (b) comprises:

repositioning the first delivery catheter with the end thereof adjacent to a proximal side of the second leaflet portion;

piercing the second leaflet portion with another needle coupled to the second suture by extending the another needle from the end of the first delivery catheter;

forming one of the second anchors on a distal side of the second leaflet portion; and

forming another of the second anchors on the proximal side of the second leaflet portion.

Example 49. The method of any example herein, particularly Example 48, wherein (b) further comprises, after the piercing, proximally retracting the first delivery catheter toward the ascending aorta.

Example 50. The method of any example herein, particularly any one of Examples 46-47, wherein (b) comprises:

positioning a second delivery catheter with an end thereof adjacent to a proximal side of the second leaflet portion, the second delivery catheter is a different catheter than the first delivery catheter;

piercing the second leaflet portion with another needle coupled to the second suture by extending the another needle from the end of the second delivery catheter;

forming one of the second anchors on a distal side of the second leaflet portion; and

forming another of the second anchors on the proximal side of the second leaflet portion.

Example 51. The method of any example herein, particularly Example 50, wherein (b) further comprises, after the piercing, proximally retracting the second delivery catheter toward the ascending aorta.

Example 52. The method of any example herein, particularly any one of Examples 46-51, wherein (c) comprises using the first delivery catheter as the coupling member that slides over the first and second sutures to urge the first and second leaflet portions together.

Example 53. The method of any example herein, particularly any one of Examples 37-52, wherein the compacting in (c) is such that the leaflet part, between the first and second anchors, folds on itself.

Example 54. The method of any example herein, particularly any one of Examples 37-53, further comprising repeating (a)-(c) with respect to another leaflet of the existing valvular structure.

Example 55. The method of any example herein, particularly any one of Examples 37-54, further comprising:

after (c), mounting a prosthetic heart valve within the existing valvular structure,

wherein the compacted leaflet part allows blood to flow through a frame of the prosthetic heart valve to a coronary artery of the heart, which would otherwise be blocked by the leaflet of the existing valvular structure.

Example 56. The method of any example herein, particularly any one of Examples 37-55, wherein said one of the first and second leaflet portions is distal to an ostium of a coronary artery of the heart.

Example 57. The method of any example herein, particularly any one of Examples 37-56, wherein said one of the first and second leaflet portions is closer to a distal attachment portion of the leaflet than a center of the existing valvular structure.

Example 58. The method of any example herein, particularly any one of Examples 37-57, wherein the existing valvular structure is a native aortic valve of the heart.

Example 59. The method of any example herein, particularly any one of Examples 37-57, wherein the existing valvular structure is of a second prosthetic heart valve previously implanted in a patient, and the mounting the prosthetic heart valve comprises installing the prosthetic heart valve within the second prosthetic heart valve.

Example 60. A system for leaflet capture, the system comprising:

a locking device comprising first and second members and having an open configuration and a closed configuration, the first and second members in the open configuration being spaced from each other by a gap, the gap being reduced or eliminated in the closed configuration,

wherein, in the open configuration, the gap between the first and second members is configured to receive a portion of a leaflet therein, and

the closed configuration is such that the leaflet portion is captured between the first and second members.

Example 61. The system of any example herein, particularly Example 60, wherein:

the first member has one or more projecting portions, and the second member has one or more recesses, each recess corresponding to one of the projecting portions,

in the open configuration, each projecting portion is separated from the corresponding recess, and

in the closed configuration, each projecting portion is inserted into the corresponding recess.

Example 62. The system of any example herein, particularly Example 61, wherein each projecting portion is constructed to pierce the leaflet portion within the gap as the locking device transitions from the open configuration to the closed configuration.

Example 63. The system of any example herein, particularly any one of Examples 60-62, wherein:

the first member has a projecting portion with one or more barbed portions,

the second member has a recess with one or more ridges therein,

in the open configuration, the projecting portion is separated from the recess, and

in the closed configuration, the projecting portion is inserted into the recess such that at least one of the barbed portions abuts at least one of the ridges so as to resist removal of the projecting portion from the recess.

Example 64. The system of any example herein, particularly any one of Examples 60-63, further comprising an actuator configured to displace the first and second members from the open configuration to the closed configuration.

Example 65. The system of any example herein, particularly Example 64, wherein the actuator is releasably coupled to the locking device so as to be disengaged therefrom once the locking device is in the closed configuration.

Example 66. The system of any example herein, particularly any one of Examples 64-65, wherein the actuator comprises a gathering portion disposed adjacent to the gap in the open configuration and configured to gather part of the leaflet into the gap as the locking device is advanced over a free end of the leaflet.

Example 67. The system of any example herein, particularly any one of Examples 64-66, wherein:

the actuator is a scissor-style actuator with a pair of arms coupled via a hinge,

one of the arms is coupled to the first member of the locking device, and

the other of the arms is coupled to the second member of the locking device.

Example 68. The system of any example herein, particularly any one of Examples 60-67, further comprising:

a gathering member disposed adjacent to the gap in the open configuration and between the locking device and the actuator,

wherein the gathering member is configured to gather part of the leaflet into the gap as the locking member is advanced over a free end of the leaflet.

Example 69. The system of any example herein, particularly Example 68, wherein the gathering member comprises part of at least one of the arms adjacent to the hinge.

Example 70. The system of any example herein, particularly Example 68, wherein the gathering member comprises a suture, wire, cable, or other flexible member extending between the first and second locking device members.

Example 71. The system of any example herein, particularly Example 68, wherein the gathering member is supported on one or more portions of the actuator.

Example 72. The system of any example herein, particularly any one of Examples 60-71, wherein the first and second members are constructed to be coupled together in the closed configuration by a snap-fit.

Example 73. A method comprising:

(a) providing a locking device in an ascending aorta of a patient, the locking device comprising first and second members and having an open configuration and a closed configuration, the first and second members in the open configuration being spaced from each other by a gap, the gap being reduced or eliminated in the closed configuration;

(b) positioning the locking device in the open configuration such that a free end of a leaflet of an existing valvular structure is disposed within the gap between the first and second members, the existing valvular structure being between the ascending aorta and a left ventricle of a heart of the patient;

(c) with the leaflet disposed within the gap, moving the locking device in the open configuration from the free end to a distal portion of the leaflet; and

(d) after (c), moving at least one of the first and second members of the locking device with respect to the other so as to transition from the open configuration to the closed configuration, thereby capturing part of the leaflet between the first and second members.

Example 74. The method of any example herein, particularly Example 73, wherein the moving in (c) is such that the leaflet folds on itself, and the captured part in (d) comprises a folded portion of the leaflet.

Example 75. The method of any example herein, particularly any one of Examples 73-74, wherein, in (d), one of the first and second members of the locking device is disposed on a proximal side of the leaflet that faces the ascending aorta, and the other of the first and second members of the locking device is disposed on a distal side of the leaflet that faces the left ventricle.

Example 76. The method of any example herein, particularly any one of Examples 73-75, further comprising:

after (d), mounting a prosthetic heart valve within the existing valvular structure,

wherein the captured leaflet part allows blood to flow through a frame of the prosthetic heart valve to a coronary artery of the heart, which would otherwise be blocked by the leaflet of the existing valvular structure.

Example 77. The method of any example herein, particularly any one of Examples 73-76, wherein:

the first member has one or more projecting portions, and the second member has one or more recesses, each recess corresponding to one of the projecting portions,

in (b) and (c), each projecting portion is separated from the corresponding recess, and

the moving in (d) is such that each projecting portion is inserted into the corresponding recess.

Example 78. The method of any example herein, particularly any one of Examples 73-76, wherein:

the first member has a projection portion with one or more barbed portions,

the second member has a recess with one or more ridges therein,

in (b) and (c), the projecting portion is separated from the recess, and

the moving in (d) is such that the projecting portion is inserted into the recess such that at least one of the barbed portions abuts at least one of the ridges so as to resist removal of the projecting portion from the recess.

Example 79. The method of any example herein, particularly any one of Examples 77-78, wherein the moving in (d) is such that at least one of the projecting portions pierces the part of the leaflet between the first and second members prior to or during insertion into the respective recess of the second member.

Example 80. The method of any example herein, particularly any one of Examples 73-79, wherein, after (d), the first and second members are coupled together in the closed configuration by a snap-fit.

Example 81. The method of any example herein, particularly any one of Examples 73-80, wherein the moving in (c) is such that one or more portions of the leaflet between the free end and the distal portion are gathered into the gap by a gathering member disposed adjacent to the gap.

Example 82. The method of any example herein, particularly any one of Examples 73-81, wherein the moving in (d) is via an actuator coupled to the locking device.

Example 83. The method of any example herein, particularly Example 82, wherein:

the actuator is a scissor-style actuator with a pair of arms coupled via a hinge,

one of the arms is coupled to the first member of the locking device, and

the other of the arms is coupled to the second member of the locking device.

Example 84. The method of any example herein, particularly Example 83, wherein the gathering member comprises a portion of the actuator that faces the gap.

Example 85. The method of any example herein, particularly any one of Examples 83-84, wherein the gathering member comprises part of at least one of the arms adjacent to the hinge.

Example 86. The method of any example herein, particularly any one of Examples 81-83, wherein the gathering member comprises a suture extending between the first and second members of the locking device.

Example 87. The method of any example herein, particularly any one of Examples 81-83, wherein the gathering member is supported on one or more portions of the actuator.

Example 88. The method of any example herein, particularly any one of Examples 82-87, further comprising, after (d), disengaging the actuator from the locking device.

Example 89. The method of any example herein, particularly any one of Examples 73-88, wherein said distal leaflet portion is distal to an ostium of a coronary artery of the heart.

Example 90. The method of any example herein, particularly any one of Examples 73-89, wherein, after (d), the locking device is disposed closer to a distal attachment portion of the leaflet than a center of the existing valvular structure.

Example 91. The method of any example herein, particularly any one of Examples 73-90, further comprising:

(e) providing a second locking device in the ascending aorta;

(f) positioning the second locking device in the open configuration such that a free end of another leaflet of the existing valvular structure is disposed within the gap between the first and second members;

(g) with the another leaflet disposed within the gap, moving the second locking device in the open configuration from the free end to a distal portion of the another leaflet; and

(h) after (g), moving at least one of the first and second members of the second locking device with respect to the other so as to transition from the open configuration to the closed configuration, thereby capturing part of the another leaflet between the first and second members.

Example 92. The method of any example herein, particularly any one of Examples 73-91, wherein the existing valvular structure is a native aortic valve of the heart.

Example 93. The method of any example herein, particularly any one of Examples 73-91, wherein the existing valvular structure is of a second prosthetic heart valve previously implanted in the patient, and the mounting the prosthetic heart valve comprises installing the prosthetic heart valve within the second prosthetic heart valve.

Example 94. A system for leaflet capture, the system comprising:

a clip member constructed to transition between a biased state and a free state, the clip member having first and second ends connected by an intermediate portion,

wherein in the free state, the intermediate portion adopts a curved configuration that defines a capture region for retaining part of a leaflet therein, and

in the biased state, the intermediate portion adopts a straight or substantially straight configuration.

Example 95. The system of any example herein, particularly Example 94, wherein the free state is such that the first and second ends are disposed adjacent to each other.

Example 96. The system of any example herein, particularly any one of Examples 94-95, wherein the clip member is formed of a flexible material.

Example 97. The system of any example herein, particularly Example 96, wherein the clip member is formed of spring metal or shape memory alloy.

Example 98. The system of any example herein, particularly Example 97, wherein the clip member is formed of steel, cobalt chromium alloy, or nickel titanium alloy.

Example 99. The system of any example herein, particularly any one of Examples 94-98, wherein at least a portion of the clip member includes a biocompatible surface coating.

Example 100. The system of any example herein, particularly any one of Examples 94-99, further comprising a delivery catheter with a lumen constructed to receive the clip member in the biased state therein, the first end of the clip member being closer to a distal end of the delivery catheter than the second end of the clip member.

Example 101. The system of any example herein, particularly Example 100, further comprising a deployment member within the delivery catheter, the deployment member being constructed to translate distally along an axial direction of the lumen to push the second member so as to deploy the clip member from the distal end of the delivery catheter.

Example 102. The system of any example herein, particularly any one of Examples 94-101, wherein in the free state, the clip member has a substantially Q-shape.

Example 103. The system of any example herein, particularly any one of Examples 94-102, wherein in the free state, a gap between the first and second ends is less than a thickness of the leaflet.

Example 104. A method comprising:

(a) providing a delivery catheter in an ascending aorta of a patient, the delivery catheter having a lumen with a clip member disposed therein, the clip member having a first end, a second end, and an intermediate portion connecting the first and second ends, the clip member being in a biased state with the first end closer to a distal end of the delivery catheter than the second end;

(b) positioning the distal end of the delivery catheter with respect to a leaflet of an existing valvular structure, the existing valvular structure being between the ascending aorta and a left ventricle of a heart of the patient;

(c) extending at least the first end of the clip member from the distal end of the delivery catheter; and

(d) after (c), releasing the clip member from the delivery catheter such that the clip member transitions to a free state so as to capture part of the leaflet,

wherein after (c) and before (d), the first end is disposed on a distal side of the leaflet,

in the free state, the intermediate portion adopts a curved configuration and defines a capture region, and

after (d), the second end is disposed on a proximal side of the leaflet, and the part of the leaflet is captured within said capture region.

Example 105. The method of any example herein, particularly Example 104, wherein, after (d), the first and second ends are disposed on opposite sides of and in contact with a first portion of the leaflet.

Example 106. The method of any example herein, particularly Example 105, wherein, after (d), the first and second ends pinch the first portion of the leaflet so as to retain the clip member in place.

Example 107. The method of any example herein, particularly any one of Examples 105-106, wherein, after (d), the part of the leaflet captured within the capture region is between the first portion of the leaflet and the free end of the leaflet.

Example 108. The method of any example herein, particularly any one of Examples 105-107, wherein:

a distance between the first end and a midpoint of the intermediate portion after (d) is less than a distance between the first portion of the leaflet and the free end of the leaflet prior to (c);

a distance between the second end and the midpoint of the intermediate portion after (d) is less than a distance between the first portion of the leaflet and the free end of the leaflet prior to (c); or

both of the above.

Example 109. The method of any example herein, particularly any one of Examples 104-108, wherein the transition to the free state in (d) is such that the part of the leaflet folds upon itself and is retained within the capture region.

Example 110. The method of any example herein, particularly any one of Examples 104-109, wherein in the biased state within the delivery catheter, the intermediate portion of the clip member has a straight or substantially straight configuration.

Example 111. The method of any example herein, particularly any one of Examples 104-110, wherein (b) is performed prior to (c).

Example 112. The method of any example herein, particularly any one of Examples 104-110, wherein (b) is performed after (c).

Example 113. The method of any example herein, particularly any one of Examples 104-110, wherein (b) and (c) are performed together.

Example 114. The method of any example herein, particularly any one of Examples 104-113, wherein (c) comprises moving a deployment member within the lumen to push the clip member distally along an axial direction of the lumen.

Example 115. The method of any example herein, particularly Example 114, wherein, during (c), the deployment member contacts the second end of the clip member.

Example 116. The method of any example herein, particularly any one of Examples 114-115, wherein (d) comprises at least one of:

moving the deployment member distally to further push the clip member out of the distal end of the delivery catheter; and

moving the delivery catheter proximally toward the ascending aorta.

Example 117. The method of any example herein, particularly any one of Examples 104-116, wherein the clip member is formed of a flexible material.

Example 118. The method of any example herein, particularly any one of Examples 104-117, wherein the clip member is formed of spring metal or shape memory alloy.

Example 119. The method of any example herein, particularly any one of Examples 104-118, wherein the clip member is formed of steel, cobalt chromium alloy, or nickel titanium alloy.

Example 120. The method of any example herein, particularly any one of Examples 104-119, wherein at least a portion of the clip member includes a biocompatible surface coating.

Example 121. The method of any example herein, particularly any one of Examples 104-120, wherein in the free state, the clip member has a substantially Q-shape.

Example 122. The method of any example herein, particularly any one of Examples 104-121, wherein a gap between the first and second ends in the free state is less than a thickness of the leaflet prior to (d).

Example 123. The method of any example herein, particularly any one of Examples 104-122, wherein the first portion of the leaflet is distal to an ostium of a coronary artery of the heart.

Example 124. The method of any example herein, particularly any one of Examples 104-123, wherein, after (d), the clip member is disposed closer to a distal attachment portion of the leaflet than a center of the existing valvular structure.

Example 125. The method of any example herein, particularly any one of Examples 104-124, further comprising, after (d):

(e) providing a second delivery catheter in the ascending aorta, the second delivery catheter having a second lumen with another clip member disposed therein, the another clip member being in the biased state with its first end closer to a distal end of the second delivery catheter that the second end;

(f) positioning the distal end of the second delivery catheter with respect to another leaflet of the existing valvular structure;

(g) extending at least the first end of the another clip member from the distal end of the second delivery catheter; and

(h) after (g), releasing the another clip member from the second delivery catheter such that the another clip member transitions to its free state so as to capture part of the another leaflet,

wherein after (g) and before (h), the first end of the another clip member is disposed on a distal side of the another leaflet,

in the free state, the intermediate portion of the another clip member adopts a curved configuration and defines another capture region, and after (h), the second end of the another clip member is disposed on a proximal side of the another leaflet, and the part of the another leaflet is captured within said capture region.

Example 126. The method of any example herein, particularly any one of Examples 104-124, further comprising, after (d):

(e) repositioning the distal end of the delivery catheter with respect to another leaflet of the existing valvular structure;

(f) extending at least a first end of another clip member from the distal end of the delivery catheter; and

(g) after (f), releasing the another clip member from the delivery catheter such that the another clip member transitions to its free state so as to capture part of the another leaflet,

wherein after (f) and before (g), the first end of the another clip member is disposed on a distal side of the another leaflet,

in the free state, the intermediate portion of the another clip member adopts a curved configuration and defines another capture region, and

after (g), the second end of the another clip member is disposed on a proximal side of the another leaflet, and the part of the another leaflet is captured within said capture region.

Example 127. The method of any example herein, particularly any one of Examples 104-126, wherein the existing valvular structure is a native aortic valve of the heart.

Example 128. The method of any example herein, particularly any one of Examples 104-127, further comprising, after (d):

mounting a prosthetic heart valve within the existing valvular structure,

wherein the captured leaflet part allows blood to flow through a frame of the prosthetic heart valve to a coronary artery of the heart, which would otherwise be blocked by the leaflet of the existing valvular structure.

Example 129. The method of any example herein, particularly Example 128, wherein the existing valvular structure is of a second prosthetic heart valve previously implanted in the patient, and the mounting the prosthetic heart valve comprises installing the prosthetic heart valve within the second prosthetic heart valve.

Example 130. A system for leaflet folding, the system comprising:

a delivery sheath constructed to be disposed within and routed through vasculature of patient;

a leaflet engagement member disposed within the delivery sheath and movable axially therein, the leaflet engagement member having a spiked tip at an axial end thereof; and

a positioning member disposed within the delivery sheath and movable axially therein, the positioning member having an atraumatic end portion.

Example 131. The system of any example herein, particularly Example 130, wherein the delivery sheath has a lumen extending therethrough, and the leaflet engagement member, the positioning member, or both are disposed within said lumen and movable axially therein.

Example 132. The system of any example herein, particularly any one of Examples 130-131, wherein the positioning member comprises a wire, cable, or catheter.

Example 133. The system of any example herein, particularly any one of Examples 130-132, wherein the atraumatic end portion of the positioning member comprises a loop.

Example 134. The system of any example herein, particularly Example 133, wherein the loop comprises a circular shape, oval shape, elliptical shape, C-shape, or J-shape.

Example 135. The system of any example herein, particularly any one of Examples 133-134, wherein the loop comprises a pair of wires coupled to each other at respective ends.

Example 136. The system of any example herein, particularly any one of Examples 130-135, wherein the spiked tip is constructed to pierce a portion of a leaflet in contact therewith.

Example 137. The system of any example herein, particularly any one of Examples 130-136, wherein the spiked tip has a barbed configuration with a laterally-extending or radially-extending (e.g., annular-shaped) base.

Example 138. The system of any example herein, particularly any one of Examples 130-137, wherein the leaflet engagement member comprises a wire or cable.

Example 139. The system of any example herein, particularly any one of Examples 130-138, wherein the leaflet engagement member is movable independent of the positioning member.

Example 140. The system of any example herein, particularly any one of Examples 130-139, further comprising:

a second delivery sheath constructed to be disposed within and routed through vasculature of patient at a same time as the delivery sheath;

a second leaflet engagement member disposed within the second delivery sheath and movable axially therein, the second leaflet engagement member having a second spiked tip at an axial end thereof; and

a second positioning member disposed within the second delivery sheath and having a second atraumatic end portion.

Example 141. A method comprising:

(a) providing a leaflet folding tool in the ascending aorta of a patient, the leaflet folding tool comprising a leaflet engagement member and a positioning member, the leaflet engagement member having a spiked tip at an axial end thereof; the positioning member having an atraumatic end portion;

(b) piercing a portion of a first leaflet of an existing valvular structure using the spiked tip;

(c) moving the leaflet engagement member and the positioning member in a direction from the ascending aorta toward the left ventricle until the atraumatic end portion of the positioning member contacts a base portion of the first leaflet, such that the pierced portion of the first leaflet is pushed toward its base portion so as to fold the first leaflet;

(d) positioning a prosthetic heart valve within a space between leaflets of the existing valvular structure; and

(e) expanding the prosthetic heart valve while the first leaflet remains folded.

Example 142. The method of any example herein, particularly Example 141, wherein, during the expanding of (e), the leaflet engagement member is removed from the pierced portion of the first leaflet.

Example 143. The method of any example herein, particularly Example 142, wherein, after the removal of the leaflet engagement member, the first leaflet remains at least partially folded due to contact with an exterior of the prosthetic heart valve.

Example 144. The method of any example herein, particularly any one of Examples 141-143, further comprising, prior to (d):

(f) providing a second leaflet folding tool in the ascending aorta of a patient, the second leaflet folding tool comprising a second leaflet engagement member and a second positioning member, the second leaflet engagement member having a second spiked tip at an axial end thereof; the second positioning member having a second atraumatic end portion;

(g) piercing a portion of a second leaflet of the existing valvular structure using the second spiked tip; and

(h) moving the second leaflet engagement member and the second positioning member in the direction from the ascending aorta toward the left ventricle until the second atraumatic end portion of the second positioning member contacts a base portion of the second leaflet, such that the pierced portion of the second leaflet is pushed toward its base portion so as to fold the second leaflet.

Example 145. The method of any example herein, particularly Example 144, wherein, during the expanding of (e), the second leaflet engagement member is removed from the pierced portion of the second leaflet.

Example 146. The method of any example herein, particularly Example 145, wherein, after the removal of the second leaflet engagement member, the second leaflet remains at least partially folded due to contact with an exterior of the prosthetic heart valve.

Example 147. The method of any example herein, particularly any one of Examples 141-146, wherein:

the leaflet folding tool further comprises a delivery sheath, the leaflet engagement member and the positioning member each being disposed at least partially within the delivery sheath and movable axially therein, and

the providing of (a) comprises moving the delivery sheath to the ascending aorta via a transcatheter aortic access route.

Example 148. The method of any example herein, particularly Example 147, wherein the transcatheter aortic access route comprises a transfemoral approach, transaxillary approach, transaortic approach, transapical approach, transcarotid approach, transseptal approach, transcaval approach, subclavian approach, radial approach, carotid approach, or any combination thereof.

Example 149. The method of any example herein, particularly any one of Examples 147-148, wherein the delivery sheath has a lumen extending therethrough, and the leaflet engagement member, the positioning member, or both are disposed within said lumen and movable axially therein.

Example 150. The method of any example herein, particularly any one of Examples 141-149, wherein the positioning member comprises a wire, cable, or catheter.

Example 151. The method of any example herein, particularly any one of Examples 141-150, wherein the atraumatic end portion of the positioning member comprises a loop.

Example 152. The method of any example herein, particularly Example 151, wherein the loop comprises a circular shape, oval shape, elliptical shape, C-shape, or J-shape.

Example 153. The method of any example herein, particularly any one of Examples 151-152, wherein the loop comprises a pair of wires coupled to each other at respective proximal ends.

Example 154. The method of any example herein, particularly any one of Examples 141-153, wherein the spiked tip has a barbed configuration with a laterally-extending or annular-shaped base.

Example 155. The method of any example herein, particularly any one of Examples 141-154, wherein the leaflet engagement member comprises a wire or cable.

Example 156. The method of any example herein, particularly any one of Examples 141-155, wherein after (e), the folded first leaflet allows blood to flow through a frame of the prosthetic heart valve to a coronary artery of the heart, which would otherwise be blocked by the first leaflet of the existing valvular structure.

Example 157. The method of any example herein, particularly any one of Examples 141-156, wherein the existing valvular structure is a native aortic valve of the heart.

Example 158. The method of any example herein, particularly any one of Examples 141-156, wherein the existing valvular structure is of a second prosthetic valve previously implanted in the patient, and the expanding the prosthetic heart valve of (e) is effective to mount the prosthetic heart valve within the second prosthetic valve.

Example 159. A system for leaflet capture, the system comprising:

a clip member constructed to be plastically deformed between an open configuration and a closed configuration, the clip member having first and second ends connected by an intermediate portion, the first and second ends in the open configuration being spaced from each other by a gap, the gap being reduced or eliminated in the closed configuration,

wherein, in the open configuration, the gap between the first and second ends is configured to receive a portion of a leaflet therein, and

the closed configuration is such that the leaflet portion is captured between the first and second ends.

Example 160. The system of any example herein, particularly Example 159, wherein the clip member is formed of metal, a metal alloy, or any combination thereof.

Example 161. The system of any example herein, particularly Example 160, wherein the clip member comprises steel or cobalt chromium alloy.

Example 162. The system of any example herein, particularly any one of Examples 159-161, wherein at least a portion of the clip member includes a biocompatible surface coating.

Example 163. The system of any example herein, particularly any one of Examples 159-162, wherein the first end, the second end, or both comprise a sharp tip for piercing a leaflet, a blunt tip for grasping the leaflet without piercing, a magnetic portion, a female member for receiving a male member of the other tip, a male member for insertion into the female member of the other tip, or any combination thereof.

Example 164. The system of any example herein, particularly any one of Examples 159-163, further comprising an actuator having a first arm and a second arm, the actuator being constructed to apply a compression force that plastically deforms the clip member from the open configuration to the closed configuration.

Example 165. The system of any example herein, particularly Example 164, wherein the actuator is releasably coupled to the clip member so as to be disengaged therefrom once the clip member is in the closed configuration.

Example 166. The system of any example herein, particularly any one of Examples 164-165, wherein the actuator comprises a tweezer, surgical tongs, or a scissor-style actuator.

Example 167. A method comprising:

(a) providing a clip member in an open configuration in an ascending aorta of a patient, the clip member having first and second ends connected together by an intermediate portion, the first and second ends in the open configuration being spaced from each other by a gap;

(b) positioning the clip member in the open configuration such that a free end of a first leaflet of an existing valvular structure extends through the gap between the first and second ends, the existing valvular structure being between the ascending aorta and a left ventricle of a heart of the patient;

(c) with the first leaflet extending through the gap, moving the clip member in the open configuration toward a distal portion of the first leaflet; and

(d) after (c), plastically deforming the clip member to a closed configuration, where the gap between the first and second ends is reduced or eliminated, such that part of the first leaflet is captured within a region enclosed by the clip member in the closed configuration.

Example 168. The method of any example herein, particularly one of Example 167, wherein the moving in (c) is such that the first leaflet folds on itself, and the captured part in (d) comprises a folded portion of the first leaflet.

Example 169. The method of any example herein, particularly any one of Examples 167-168, further comprising:

after (d), mounting a prosthetic heart valve within the existing valvular structure,

wherein the captured part of the first leaflet allows blood to flow through a frame of the prosthetic heart valve to a coronary artery of the heart, which would otherwise be blocked by the first leaflet.

Example 170. The method of any example herein, particularly any one of Examples 167-169, wherein, after (d), the first and second ends are disposed on opposite sides of and in contact with a first portion of the first leaflet, or the first and second ends pierce through opposite side of the first portion of the first leaflet.

Example 171. The method of any example herein, particularly Example 170, wherein, after (d), the part of the first leaflet captured by the clip member is between the first portion and the free end of the first leaflet.

Example 172. The method of any example herein, particularly any one of Examples 170-171, wherein:

a distance between the first end and a midpoint of the intermediate portion after (d) is less than a distance between the first portion and the free end of the first leaflet prior to (c);

a distance between the second end and the midpoint of the intermediate portion after (d) is less than a distance between the first portion and the free end of the first leaflet prior to (c); or

both of the above.

Example 173. The method of any example herein, particularly any one of Examples 167-172, wherein the moving in (c) is via an actuator coupled to the clip member.

Example 174. The method of any example herein, particularly Example 173, wherein the actuator has a first arm and a second arm, and the actuator is constructed to apply a compression force that plastically deforms the clip member to the closed configuration in (d).

Example 175. The method of any example herein, particularly Example 174, further comprising, after (d), disengaging the actuator from the clip member is in the closed configuration, and removing the actuator from the ascending aorta.

Example 176. The method of any example herein, particularly any one of Examples 173-175, wherein the actuator comprises tweezers, surgical tongs, or a scissor-style actuator.

Example 177. The method of any example herein, particularly any one of Examples 167-176, wherein the clip member is formed of metal, a metal alloy, or any combination thereof.

Example 178. The method of any example herein, particularly Example 177, wherein the clip member comprises steel or cobalt chromium alloy.

Example 179. The method of any example herein, particularly any one of Examples 167-178, wherein at least a portion of the clip member includes a biocompatible surface coating.

Example 180. The method of any example herein, particularly any one of Examples 167-179, wherein the first end, the second end, or both comprise a sharp tip for piercing a leaflet, a blunt tip for grasping the leaflet without piercing, a magnetic portion, a female member for receiving a male member of the other tip, a male member for insertion into the female member of the other tip, or any combination thereof.

Example 181. The method of any example herein, particularly any one of Examples 173-180, wherein:

the providing of (a) comprises moving a delivery sheath to the ascending aorta via a transcatheter aortic access route, and

the actuator, the clip member, or both are disposed within the delivery sheath.

Example 182. The method of any example herein, particularly Example 181, wherein the transcatheter aortic access route comprises a transfemoral approach, transaxillary approach, transaortic approach, transapical approach, transcarotid approach, transseptal approach, transcaval approach, subclavian approach, radial approach, carotid approach, or any combination thereof.

Example 183. The method of any example herein, particularly any one of Examples 167-182, wherein the existing valvular structure is a native aortic valve of the heart.

Example 184. The method of any example herein, particularly any one of Examples 167-182, wherein the existing valvular structure is of a second prosthetic valve previously implanted in the patient, and the expanding the prosthetic heart valve of (e) is effective to mount the prosthetic heart valve within the second prosthetic valve.

Example 185. A method comprising:

providing means for leaflet capture within an ascending aorta of a patient;

expanding a prosthetic heart valve to implant the prosthetic heart valve within an existing valvular structure located between the ascending aorta and the left ventricle of the patient; and

prior to or during the expanding of the prosthetic heart valve, using the means for leaflet capture to capture, fold, compact, or otherwise position one or more leaflets of the existing valvular structure distal to an ostium of a coronary artery of the heart, such that blood is allowed to flow through a frame of the prosthetic heart valve to said coronary artery after the expanding.

Example 186. The method of any example herein, particularly Example 185, wherein the means for leaflet capture is part of or attached to the prosthetic heart valve.

Example 187. The method of any example herein, particularly any one of Examples 185-186, wherein the means for leaflet capture comprises one or more leaflet capture members disposed along an exterior of the prosthetic heart valve frame and coupled at opposite ends thereof to the frame, the leaflet capture member extending along an axial direction of the frame, the leaflet capture member comprising a distal portion, a proximal portion, and an intermediate portion between and coupled to the distal and proximal portions by respective connecting portions, the leaflet capture member being configured to change shape upon expansion of the frame for implantation so as to capture a free end of a leaflet of the existing valvular structure between the intermediate portion and the distal portion.

Example 188. The method of example herein, particularly any one of Examples 185-187, wherein the means for leaflet capture is provided to the ascending aorta by a delivery sheath or catheter.

Example 189. The method of example herein, particularly any one of Examples 185-188, wherein the means for leaflet capture comprises a suture passing through a first portion of a leaflet of the existing valvular structure, and the using the means for leaflet capture comprises:

forming a knot by tying a first portion of the suture to a second portion of the suture, such that part of the leaflet between the first portion of the leaflet and a free end of the leaflet is disposed within a loop formed between the tied first and second suture portions; and

sliding at least one of the knot and the suture with respect to the other, so as to reduce a size of the loop, thereby compacting the leaflet part by moving the leaflet free end toward the first portion of the leaflet.

Example 190. The method of any example herein, particularly Example 189, wherein the sliding comprises moving a sliding member distally along the suture to push the knot toward the first leaflet portion.

Example 191. The method of any example herein, particularly Example 190, wherein the sliding member comprises a portion of a delivery catheter, or the sliding member is extended from an end of the delivery catheter.

Example 192. The method of any example herein, particularly any one of Examples 185-191, wherein the means for leaflet capture comprises a first anchor for a first suture formed at a first portion of a leaflet of the existing valvular structure and a second anchor for a second suture formed at a second portion of the leaflet, and the using the means for leaflet capture comprises:

pulling one of the first and second leaflet portions toward the other of the first and second leaflet portions so as to compact part of the leaflet disposed between the first and second anchors.

Example 193. The method of any example herein, particularly Example 192, wherein the pulling comprises distally sliding a coupling member along the first and second sutures from the ascending aorta toward the leaflet.

Example 194. The method of any example herein, particularly Example 193, wherein the coupling member has one or more conduits that define a spacing between the first and second sutures extending therethrough, the spacing being less than a distance along the leaflet between the first and second leaflet portions.

Example 195. The method of any example herein, particularly any one of Examples 185-194, wherein the means for leaflet capture comprises a locking device, the locking device comprising first and second members and having an open configuration and a closed configuration, the first and second members in the open configuration being spaced from each other by a gap, the gap being reduced or eliminated in the closed configuration, and

the using the means for leaflet capture comprises:
positioning the locking device in the open configuration such that a free end of a leaflet of an existing valvular structure is disposed within the gap between the first and second members, the existing valvular structure being between the ascending aorta and a left ventricle of a heart of the patient;
with the leaflet disposed within the gap, moving the locking device in the open configuration from the free end to a distal portion of the leaflet; and
moving at least one of the first and second members of the locking device with respect to the other so as to transition from the open configuration to the closed configuration, thereby capturing part of the leaflet between the first and second members.

Example 196. The method of any example herein, particularly Example 195, wherein the moving the locking device in the open configuration is such that the leaflet folds on itself, and the captured part comprises a folded portion of the leaflet.

Example 197. The method of any example herein, particularly any one of Examples 195-196, wherein:

the first member has a projection portion with one or more barbed portions,

the second member has a recess with one or more ridges therein,

in the positioning the locking device and the moving the locking device in the open configuration, the projecting portion is separated from the recess, and

the moving at least one of the first and second members is such that the projecting portion is inserted into the recess such that at least one of the barbed portions abuts at least one of the ridges so as to resist removal of the projecting portion from the recess.

Example 198. The method of any example herein, particularly any one of Examples 195-197, wherein:

the moving at least one of the first and second members is via an actuator coupled to the locking device,

the actuator is a scissor-style actuator with a pair of arms coupled via a hinge,

one of the arms is coupled to the first member of the locking device, and

the other of the arms is coupled to the second member of the locking device.

Example 199. The method of any example herein, particularly any one of Examples 185-198, wherein the means for leaflet capture comprises a clip member constructed to transition between a biased state and a free state, the clip member having first and second ends connected by an intermediate portion, wherein in the free state, the intermediate portion adopts a curved configuration that defines a capture region for retaining part of a leaflet therein, and in the biased state, the intermediate portion adopts a straight or substantially straight configuration, and

the using the means for leaflet capture comprises:
extending at least the first end of the clip member in the biased state from a distal end of a delivery catheter positioned with respect to a leaflet of the existing valvular structure; and
releasing the clip member from the delivery catheter such that the clip member transitions to the free state so as to capture part of the leaflet within the capture region.

Example 200. The method of any example herein, particularly Example 199, wherein the clip member is formed of spring metal or shape memory alloy.

Example 201. The method of any example herein, particularly any one of Examples 185-200, wherein the means for leaflet capture comprises a leaflet folding tool, the leaflet folding tool comprising a leaflet engagement member and a positioning member, the leaflet engagement member having a spiked tip at an axial end thereof; the positioning member having an atraumatic end portion, and

the using the means for leaflet capture comprises:
piercing a portion of a leaflet of the existing valvular structure using the spiked tip; and
   moving the leaflet engagement member and the positioning member in a direction from the ascending aorta toward the left ventricle until the atraumatic end portion of the positioning member contacts a base portion of the leaflet, such that the pierced portion of the leaflet is pushed toward its base portion so as to fold the leaflet.

Example 202. The method of any example herein, particularly Example 201, wherein the leaflet engagement member is removed from the pierced portion of the leaflet after the prosthetic heart valve has been expanded from an initial crimped configuration but before the prosthetic heart valve has been expanded to its full implanted size.

Example 203. The method of any example herein, particularly any one of Examples 201-202, wherein, after the removal of the leaflet engagement member, the leaflet remains at least partially folded due to contact with an exterior of the prosthetic heart valve.

Example 204. The method of any example herein, particularly any one of Examples 201-203, wherein:

the positioning member comprises a wire, cable or catheter;

the atraumatic end portion of the positioning member comprises a loop;

the leaflet engagement member comprises a wire or cable;

the spiked tip of the leaflet engagement member has a barbed configuration with a laterally-extending or radially-extending base; or

any combination of the above.

Example 205. The method of any example herein, particularly any one of Examples 185-204, wherein the means for leaflet capture comprises a clip member constructed to be plastically deformed between an open configuration and a closed configuration, the clip member having first and second ends connected by an intermediate portion, the first and second ends in the open configuration being spaced from each other by a gap, the gap being reduced or eliminated in the closed configuration, and

the using the means for leaflet capture comprises:
positioning the clip member in the open configuration such that a free end of a leaflet of the existing valvular structure extends through the gap between the first and second ends;
with the leaflet extending through the gap, moving the clip member in the open configuration toward a distal portion of the leaflet; and
plastically deforming the clip member to the closed configuration, such that part of the leaflet is captured within a region enclosed by the clip member in the closed configuration.

Example 206. The method of any example herein, particularly Example 205, wherein the moving the clip member is such that the leaflet folds on itself, and the captured part comprises a folded portion of the leaflet.

Example 207. The method of any example herein, particularly any one of Examples 205-206, wherein the plastically deforming the clip member to the closed configuration is by an actuator having first and second arms constructed to apply a compression force to the clip member.

Example 208. The method of any example herein, particularly any one of Examples 185-207, wherein the providing comprises moving a delivery sheath, which has the means for leaflet capture therein or thereon, to the ascending aorta via a transcatheter aortic access route.

Example 209. The method of any example herein, particularly any one of Examples 185-207, wherein the existing valvular structure is a native aortic valve of the heart.

Example 210. The method of any example herein, particularly any one of Examples 185-207, wherein the existing valvular structure is of a second prosthetic heart valve previously implanted in the patient, and the mounting the prosthetic heart valve comprises installing the prosthetic heart valve within the second prosthetic heart valve.

### CONCLUSION

All features described herein are independent of one another and, except where structurally impossible, can be used in combination with any other feature described herein. For example, the delivery system of FIG. 2C or the prosthetic valve delivery described with respect to FIG. 4G can be used to implant the prosthetic heart valve in any other of the disclosed examples. In another example, aspects of clip member 700 described with respect to FIGS. 7A-7D can be applied to the plastically-deformed clip member 1000 of FIGS. 10A-11E, and vice versa. Indeed, any of the features illustrated or described with respect to FIGS. 1-11E and Clauses 1-210 can be combined with any other feature illustrated or described with respect to FIGS. 1-11E and Clauses 1-210 to provide systems, methods, devices, and embodiments not otherwise illustrated or specifically described herein.

In view of the many possible embodiments to which the principles of the disclosed technology may be applied, it should be recognized that the illustrated embodiments are only preferred examples and should not be taken as limiting the scope of the disclosed technology.

Rather, the scope is defined by the following claims. We therefore claim all that comes within the scope and spirit of these claims.

1. A prosthetic heart valve comprising:
an annular frame that is expandable from a crimped state to a deployed state;
a first valvular structure formed by a leaflet assembly, the leaflet assembly comprising a plurality of leaflets, the first valvular structure being disposed within and attached to the annular frame; and
a leaflet capture member disposed along an exterior of the annular frame and coupled at opposite ends thereof to the annular frame, the leaflet capture member extending along an axial direction of the annular frame, the leaflet capture member comprising a distal portion, a proximal portion, and an intermediate portion between and coupled to the distal and proximal portions by respective connecting portions,
wherein the leaflet capture member is configured to change shape upon expansion of the annular frame to the deployed state so as to capture a free end of a leaflet of an existing second valvular structure between the intermediate portion and the distal portion.

2. The prosthetic heart valve of claim 1, further comprising additional leaflet capture members disposed at different locations along a circumference of the annular frame.

3. The prosthetic heart valve of claim 1, wherein each leaflet capture member comprises a substantially straight bar extending along the axial direction prior to expansion of the annular frame.

4. The prosthetic heart valve of claim 1, wherein at least the proximal portion of each leaflet capture member has an open cell structure that allows blood to flow therethrough.

5. The prosthetic heart valve of claim 1, wherein at least one of the connecting portions comprises a weakened region.

6. The prosthetic heart valve of claim 1, wherein at least one of the connecting portions comprises a region with a narrowed cross-section compared to the distal portion, the proximal portion, and/or the intermediate portion.

7. The prosthetic heart valve of claim 1, wherein at least one of the connecting portions comprises a notched or grooved region.

8. The prosthetic heart valve of claim 1, wherein the leaflet capture member is formed of metal or a biocompatible polymer.

9. The prosthetic heart valve of claim 1, wherein the leaflet capture member is configured such that:
prior to expansion of the annular frame, a first connecting portion that couples the proximal portion to the intermediate portion is disposed proximal to a second connecting portion that couples the distal portion to the intermediate portion; and
after expansion of the annular frame, the first connecting portion is disposed distal to the second connecting portion.

10. A system for leaflet capture, the system comprising:
a locking device comprising first and second members and having an open configuration and a closed configuration, the first and second members in the open configuration being spaced from each other by a gap, the gap being reduced or eliminated in the closed configuration,
wherein, in the open configuration, the gap between the first and second members is configured to receive a portion of a leaflet therein, and
the closed configuration is such that the leaflet portion is captured between the first and second members.

11. The system of claim 10, wherein:
the first member has one or more projecting portions, and the second member has one or more recesses, each recess corresponding to one of the projecting portions,
in the open configuration, each projecting portion is separated from the corresponding recess, and
in the closed configuration, each projecting portion is inserted into the corresponding recess.

12. The system of claim 11, wherein each projecting portion is constructed to pierce the leaflet portion within the gap as the locking device transitions from the open configuration to the closed configuration.

13. The system of claim 10, wherein:
the first member has a projecting portion with one or more barbed portions,
the second member has a recess with one or more ridges therein,
in the open configuration, the projecting portion is separated from the recess, and
in the closed configuration, the projecting portion is inserted into the recess such that at least one of the barbed portions abuts at least one of the ridges so as to resist removal of the projecting portion from the recess.

14. The system of claim 10, further comprising an actuator configured to displace the first and second members from the open configuration to the closed configuration.

15. The system of claim 14, wherein the actuator is releasably coupled to the locking device so as to be disengaged therefrom once the locking device is in the closed configuration.

16. The system of claim 14, wherein the actuator comprises a gathering portion disposed adjacent to the gap in the open configuration and configured to gather part of the leaflet into the gap as the locking device is advanced over a free end of the leaflet.

17. The system of claim 14, wherein:
the actuator is a scissor-style actuator with a pair of arms coupled via a hinge,
one of the arms is coupled to the first member of the locking device, and
the other of the arms is coupled to the second member of the locking device.

18. The system of claim 10, further comprising:
a gathering member disposed adjacent to the gap in the open configuration and between the locking device and the actuator,
wherein the gathering member is configured to gather part of the leaflet into the gap as the locking member is advanced over a free end of the leaflet.

19. The system of claim 18, wherein the gathering member comprises part of at least one of the arms adjacent to the hinge.

20. The system of claim 18, wherein the gathering member comprises a suture, wire, cable, or other flexible member extending between the first and second locking device members.
